SEQUENCE LISTING (14 sequences)

SEQ ID NO: 1 - DNA, 64 bp, Ocimum basilicum
  source 1..64: mol_type = genomic DNA
  variation 7
gacattttgcaaactggaaaaacgattttcatcagctcaactttacagatcggaagagcggttc

SEQ ID NO: 2 - DNA, 64 bp, Ocimum basilicum
  source 1..64: mol_type = genomic DNA
  variation 5
gtctatccagaagcagaatgaggcgatcaccgggaagattttcttgctggacgatctccctctt

SEQ ID NO: 3 - DNA, 64 bp, Ocimum basilicum
  source 1..64: mol_type = genomic DNA
  variation 48
aaaacggaatctagggttttcagcacttcttttgctagtttgggtgacgaaacaacgattacag

SEQ ID NO: 4 - DNA, 64 bp, Ocimum basilicum
  source 1..64: mol_type = genomic DNA
  variation 4
aaggtggaatctagggttttgagcacttctttcgcgagttcgggggaagaaatgacgattacag

SEQ ID NO: 5 - DNA, 64 bp, Ocimum basilicum
  source 1..64: mol_type = genomic DNA
  variation 19
atgaagcggacttggatcctcacgtcggtggtgagtttattctatttaccgtatatgaacagtt

SEQ ID NO: 6 - DNA, 56 bp, Ocimum basilicum
  source 1..56: mol_type = genomic DNA
  variation 8
ttgaataatcattttctttccaaaattgttgagcagttggctgcatagtcaattac

SEQ ID NO: 7 - DNA, 64 bp, Ocimum basilicum
  source 1..64: mol_type = genomic DNA
  variation 29
cattagtccccgaagctccggatgtgaatatatggtttttctggaaagaaagcgatcgaaaatc

SEQ ID NO: 8 - DNA, 64 bp, Ocimum basilicum
  source 1..64: mol_type = genomic DNA
  variation 55
atagctgaaccccatcgacaacaccatctgcatgctgctgctcagaactatcggtcccagctcc

SEQ ID NO: 9 - DNA, 64 bp, Ocimum basilicum
  source 1..64: mol_type = genomic DNA
  variation 41
tagccgctgccgcttgagaagctgcattagttgtttcagctgcaactaaatcttcagcaaccag

SEQ ID NO: 10 - DNA, 64 bp, Ocimum basilicum
  source 1..64: mol_type = genomic DNA
  variation 11
tagaaggagacccatattcatcaaaaagtcgcataagatctccaaactggccgtgcaaatcacc

SEQ ID NO: 11 - DNA, 64 bp, Ocimum basilicum
  source 1..64: mol_type = genomic DNA
  variation 6
cagcaggagaaggtgtatggtggtgaacagagtacgagtgatccctcttcatcaaatatatcta

SEQ ID NO: 12 - DNA, 64 bp, Ocimum basilicum
  source 1..64: mol_type = genomic DNA
  variation 41
aaagagaaaaggttatgggcgacccaaagtccataattccggcagcatttgtttcattcaaaag

SEQ ID NO: 13 - DNA, 64 bp, Ocimum basilicum
  source 1..64: mol_type = genomic DNA
  variation 7
aaaaaataaataaataaatccagataagaacaaataaactaattgtagagcggtgagcaaggtc

SEQ ID NO: 14 - DNA, 64 bp, Ocimum basilicum
  source 1..64: mol_type = genomic DNA
  variation 7
aaatggccaagtcctgcaaaggcctggctatggagctcgtcaaatgtctcagtgagtccgactg

(19) United States
(12) Patent Application Publication
(10) Pub. No.: US 2025/0255239 A1
(43) Pub. Date: Aug. 14, 2025

(54) NOVEL MARKERS COMBINATION IN SWEET BASIL FOR DISEASE RESISTANCES

(71) Applicant: BAR-ILAN UNIVERSITY, Ramat Gan (IL)

(72) Inventors: Yigal COHEN, Kiryat Ono (IL); Yariv BEN NAIM, Yehud (IL)

(73) Assignee: BAR-ILAN UNIVERSITY, Ramat Gan (IL)

(21) Appl. No.: 19/176,968

(22) Filed: Apr. 11, 2025

Related U.S. Application Data
(62) Division of application No. 18/681,473, filed on Feb. 5, 2024.
(60) Provisional application No. 63/367,353, filed on Jun. 30, 2022.

(51) Int. Cl. A01H 6/50; A01H 1/00; A01H 5/12; C12Q 1/6869; C12Q 1/6895

(52) U.S. Cl. CPC A01H 6/506; A01H 1/1255; A01H 5/12; C12Q 1/6869; C12Q 1/6895; C12Q 2600/13; C12Q 2600/156

(57) ABSTRACT

The present invention discloses a disease resistant cultivated basil plant and/or seed comprising a genomic sequence having an introgressed basil downy mildew (BDM) resistance or tolerance associated haplotype derived from Ocimum americanum. A method for producing the same is also disclosed.

### CROSS-REFERENCE TO RELATED APPLICATIONS

This application is a division of U.S. application Ser. No. 18/681,473 filed Feb. 5, 2024, which is the U.S. national phase of PCT Application No. PCT/IL2023/050064 filed Jan. 19, 2023, which, in turn, claims priority from U.S. Provisional Patent Application No. 63/367,353, filed on Jun. 30, 2022, the disclosures of which are incorporated by reference in their entirety herein.

### REFERENCE TO AN ELECTRONIC SEQUENCE LISTING

The contents of the electronic sequence listing (MEITP0133PUSA_Seq List st26 Jan. 19, 2023 12777062.1.xml; Size: 16,006 bytes; and Date of Creation: Jan. 29, 2024) is herein incorporated by reference in its entirety.

### FIELD OF THE INVENTION

The present disclosure relates to conferring desirable disease resistance traits in basil plants. More particularly, the current invention pertains to methods for producing basil plants with improved tolerance to pathogens.

### BACKGROUND OF THE INVENTION

Downy mildew caused by the oomycete Peronospora belbahrii (Thines) is currently the most devastating foliar disease of sweet basil. Basil is one of the most consumed fresh herbs, with extreme regulation of pesticide residues. Therefore, most effective measures for disease control are undertaken to incorporating resistance genes into commercial cultivars by classical breeding or via interspecies hybridization. Potential sources of resistance against basil downy mildew (BDM) do not occur among commercial sweet basil varieties but are common in wild Ocimum species.

F1 hybrids derived from a cross between the wild Ocimum accessions O. americanum var. americanum, O. americanum var. pilosum, O. kilimandscharicum and O. tenuiflorum (Syn-O. sanctum or ‘Tulsi’) with commercial basils were highly resistant, signifying them as potential sources for dominant resistance genes. However, all those hybrids were sterile.

Moreover, all these wild genotypes and their hybrids represented exotic basils, differing greatly in morphology, aromas and taste which do not meet the culinary market requirements. The sexual incompatibility of F1 hybrids occurs due to differences in ploidy and/or lack of chromosome homology (Ben-Naim et al. 2015; Ben-Naim et al. 2018; Carovic-Stanko et al. 2010; Paton and Putievsky 1996; Mukherjee et al. 2005).

In genetic studies, segregating populations may reveal new phenotypes which do not exist in the original parents due to genetic complementation. Outcrossing is an adaptive procedure because it facilitates complementation by the masking of deleterious recessive alleles (Bernstein et al. 1985).

For decades, dominance, overdominance and dominant complimentary interaction models have been suggested to explain the genetic basis of gene complementation and heterosis. The dominance model of heterosis is based on the hypothesis that slightly deleterious recessive alleles in many genes are complemented by superior dominant alleles in the hybrid (Hochholdinger and Hoecker, 2007; Chen et al. 2010; Yang et al. 2017). The overdominance model attributes the superiority of hybrids to heterozygosity itself and hence allelic interactions at multiple loci (Schnable and Springer 2013; Chen et al. 2010). However, the situation is further complicated when the analysis of heterosis is extended to polyploidy, which exhibit progressive heterosis that cannot be explained by the dominance model. Progressive heterosis describes the scenario in which hybrid vigor generally increases with the number of distinct genomes present in allopolyploids. A similar observation has been made in wheat, where tetraploids and hexaploids display progressive increases in heterosis, even though the ratio of positive and negative alleles has not been altered in polypoid versus diploid plants (Suzuki, et al 2015; Chen 2010). Several examples of overdominance were also reported in several plant species where a heterotic trait was conditioned by a “single gene” e.g. Arabidopsis (erecta and augustifolia) (Chen 2010, Chen 2013), maize (pl) (Baldauf et al. 2018: Yang et al., 2017; Guo et al. 2010) and tomato (SFT) (Krieger et al. 2010).

Whereas a gene for BDM resistance exists in the wild inedible Ocimum ammericanum, no resistance gene exists in commercially available Ocimum basilicum. Therefore, due to the edible plants being susceptible to BDM, farmers continue to suffer severe losses due to downy mildew.

It is therefore a long felt need to develop means and methods to produce basil plants for commercial use with advanced resistance to various disease causing pathogens.

### SUMMARY OF THE INVENTION

It is therefore one object of the present invention to disclose a disease resistant cultivated basil plant and/or seed, wherein said basil plant and/or seed comprising a genomic sequence having an introgressed basil downy mildew (BDM) resistance or tolerance associated haplotype, said haplotype is derived from Ocimum americanum or any hybrid thereof.

It is a further object of the present invention to disclose the disease resistant cultivated basil plant and/or seed as defined above, wherein said introgressed BDM resistance or tolerance associated haplotype is derived from Ocimum americanum var. americanum and/or from Ocimum americanum var. pilosum.

It is a further object of the present invention to disclose the disease resistant cultivated basil plant and/or seed as defined in any of the above, wherein said introgressed BDM resistance or tolerance associated haplotype comprises at least one of: a single nucleotide polymorphism (SNP) of nucleotide T at position 7 of a sequence comprising at least 80% identity to SEQ ID NO:1, SNP of nucleotide A at position 5 of a sequence comprising at least 80% identity to SEQ ID NO:2, SNP of nucleotide C at position 48 of a sequence comprising at least 80% identity to SEQ ID NO:3, SNP of nucleotide G at position 4 of a sequence comprising at least 80% identity to SEQ ID NO:4, SNP of nucleotide T at position 19 of a sequence comprising at least 80% identity to SEQ ID NO: 5, SNP of nucleotide A at position 8 of a sequence comprising at least 80% identity to SEQ ID NO: 6, SNP of nucleotide T at position 29 of a sequence comprising at least 80% identity to SEQ ID NO:7, SNP of nucleotide T at position 55 of a sequence comprising at least 80% identity to SEQ ID NO:8, SNP of nucleotide T at position 41 of a sequence comprising at least 80% identity to SEQ ID NO:9, SNP of nucleotide C at position 11 of a sequence comprising at least 80% identity to SEQ ID NO: 10, SNP of nucleotide G at position 6 of a sequence comprising at least 80% identity to SEQ ID NO:11 or any combination thereof.

It is a further object of the present invention to disclose the disease resistant cultivated basil plant and/or seed as defined in any of the above, wherein said introgressed BDM resistance or tolerance associated haplotype comprises at least one nucleic acid sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO: 8, SEQ ID NO:9, SEQ ID NO:10, SEQ ID NO:11 or any combination thereof.

It is a further object of the present invention to disclose the disease resistant cultivated basil plant and/or seed as defined in any of the above, wherein said introgressed BDM resistance or tolerance associated haplotype is associated with conferring resistance or tolerance to BDM race-0, BDM race-1 or a combination thereof.

It is a further object of the present invention to disclose the disease resistant cultivated basil plant and/or seed as defined above, wherein said introgressed SEQ ID NO:1, SEQ ID NO:2, SEQ ID NO: 3, SEQ ID NO:4, SEQ ID NO:5, SEQ ID NO:6 and/or SEQ ID NO:7, is associated with resistance or tolerance to BDM race-0.

It is a further object of the present invention to disclose the disease resistant cultivated basil plant and/or seed as defined above, wherein said introgressed SEQ ID NO:8, SEQ ID NO:9, SEQ ID NO: 10 and/or SEQ ID NO:11 is associated with resistance or tolerance to BDM race-1.

It is a further object of the present invention to disclose the disease resistant cultivated basil plant and/or seed as defined above, wherein the haplotype comprising a combination of one or more of introgressed SEQ ID NO:1, SEQ ID NO:2, SEQ ID NO:3, SEQ ID NO:4, SEQ ID NO:5, SEQ ID NO: 6 and/or SEQ ID NO:7, together with one or more of SEQ ID NO:8, SEQ ID NO:9, SEQ ID NO: 10 and/or SEQ ID NO:11 is associated with synergistic effect on resistance or tolerance to BDM, specifically to BDM race-0 and BDM race-1, as compared to the effect associated with each of the sequences separately.

It is a further object of the present invention to disclose the disease resistant cultivated basil plant and/or seed as defined in any of the above, wherein said basil plant exhibit a synergistic effect with respect to resistance or tolerance to BDM, specifically to BDM race-0 and BDM race-1.

It is a further object of the present invention to disclose the disease resistant cultivated basil plant and/or seed as defined in any of the above, wherein said resistance or tolerance to BDM is higher relative to a basil plant of the same species lacking the at least one introgressed nucleic acid sequences associated with resistance or tolerance to BDM race-0 or BDM race-1.

It is a further object of the present invention to disclose the disease resistant cultivated basil plant and/or seed as defined in any of the above, wherein the cultivated basil plant and/or seed is selected from the group consisting of Ocimum kilimandscharicum (O. kilimandscharicum), O. tenuiflorum, O. basilicum, O. basilicum var. anisatum, O. basilicum var. thyrsiflorum, O. basilicum var. citrodorum and O. x citrodorum (Syn O. americanum Lemon Types), O. basilicum var. minimum, Ocimum basilicum var. siam-queen, Ocimum basilicum cv. Perrie, Ocimum basilicum L. and hybrids thereof.

It is a further object of the present invention to disclose the disease resistant cultivated basil plant and/or seed as defined in any of the above, wherein the cultivated basil plant and/or seed is an Ocimum basilicum plant and/or seed or any hybrid thereof.

It is a further object of the present invention to disclose the disease resistant cultivated basil plant and/or seed as defined in any of the above, wherein the cultivated basil plant and/or seed is Ocimum basilicum or any hybrid thereof and the introgressed BDM resistance or tolerance associated haplotype is derived from Ocimum americanum or any hybrid thereof.

It is a further object of the present invention to disclose the disease resistant cultivated basil plant and/or seed as defined in any of the above, wherein said cultivated basil plant and/or seed is Ocimum basilicum or any hybrid thereof, and the genome of said plant and/or seed comprises a haplotype comprising at least one of: a single nucleotide polymorphism (SNP) of nucleotide G at position 41 of a sequence comprising at least 80% identity to SEQ ID NO:12, SNP of nucleotide T at position 7 of a sequence comprising at least 80% identity to SEQ ID NO:13 and SNP of nucleotide C at position 7 of a sequence comprising at least 80% identity to SEQ ID NO: 14 or any combination thereof.

It is a further object of the present invention to disclose the disease resistant cultivated basil plant and/or seed as defined in any of the above, wherein said cultivated basil plant and/or seed is an Ocimum basilicum or any hybrid thereof, and the genome of said plant and/or seed comprises at least one nucleic acid sequence selected from SEQ ID NO:12, SEQ ID NO:13 and SEQ ID NO:14 or any combination thereof.

It is a further object of the present invention to disclose the disease resistant cultivated basil plant and/or seed as defined in any of the above, wherein the cultivated basil plant and/or seed comprises a haplotype comprising: (a) one or more of SEQ ID NO: 12-14, (b) one or more of SEQ ID NO:1-7, and (c) one or more of SEQ ID NO:8-11 or any combination thereof.

It is a further object of the present invention to disclose the disease resistant cultivated basil plant and/or seed as defined in any of the above, wherein the cultivated basil plant and/or seed is an Ocimum basilicum or any hybrid thereof, having double BDM resistance or tolerance to BDM race-0 and BDM race-1.

It is a further object of the present invention to disclose the disease resistant cultivated basil plant and/or seed as defined in any of the above, wherein said resistant cultivated basil plant is fertile.

It is a further object of the present invention to disclose the disease resistant cultivated basil plant and/or seed as defined in any of the above, wherein the plant and/or seed is homozygous or heterozygous to any one of SEQ ID NO: 1-14, or any combination thereof.

It is a further object of the present invention to disclose a disease resistant cultivated basil plant and/or seed, wherein said basil plant and/or seed comprising a genomic sequence having an introgressed basil downy mildew (BDM) resistance or tolerance associated haplotype, wherein said haplotype comprises a) a single nucleotide polymorphism (SNP) of nucleotide T at position 7 of a sequence comprising at least 80% identity to SEQ ID NO: 1, SNP of nucleotide A at position 5 of a sequence comprising at least 80% identity to SEQ ID NO:2, SNP of nucleotide C at position 48 of a sequence comprising at least 80% identity to SEQ ID NO:3, SNP of nucleotide G at position 4 of a sequence comprising at least 80% identity to SEQ ID NO:4, SNP of nucleotide T at position 19 of a sequence comprising at least 80% identity to SEQ ID NO:5, SNP of nucleotide A at position 8 of a sequence comprising at least 80% identity to SEQ ID NO:6, SNP of nucleotide T at position 29 of a sequence comprising at least 80% identity to SEQ ID NO:7 or any combination thereof, associated with resistance to BDM race-0; and b) SNP of nucleotide T at position 55 of a sequence comprising at least 80% identity to SEQ ID NO:8, SNP of nucleotide T at position 41 of a sequence comprising at least 80% identity to SEQ ID NO:9, SNP of nucleotide Cat position 11 of a sequence comprising at least 80% identity to SEQ ID NO:10, or SNP of nucleotide G at position 6 of a sequence comprising at least 80% identity to SEQ ID NO:11 or any combination thereof, associated with resistance to BDM race-1; and wherein said cultivated basil plant exhibits synergistic effect with respect to BDM resistance, as compared to the effect exhibited by a basil plant comprising only one of BDM race-0 or BDM race-1 associated haplotype. It is a further object of the present invention to disclose the disease resistant cultivated basil plant and/or seed as defined above, wherein said cultivated basil plant and/or seed is Ocimum basilicum or any hybrid thereof, and the genome of said plant and/or seed comprises a haplotype comprising at least one of: a single nucleotide polymorphism (SNP) of nucleotide G at position 41 of a sequence comprising at least 80% identity to SEQ ID NO:12, SNP of nucleotide T at position 7 of a sequence comprising at least 80% identity to SEQ ID NO:13 and SNP of nucleotide C at position 7 of a sequence comprising at least 80% identity to SEQ ID NO: 14 or any combination thereof.

It is a further object of the present invention to disclose a plant part comprising at least one regenerable cell, pollen, ovule, fruit or seed of the disease resistant cultivated basil plant as defined in any of the above.

It is a further object of the present invention to disclose a tissue culture of regenerable cells, protoplasts or callus obtained from the disease resistant cultivated basil plant, seed or plant part as defined in any of above.

It is a further object of the present invention to disclose a method for producing a disease resistant cultivated basil plant and/or seed, the method comprising: a) selecting a first Ocimum americanum basil plant exhibiting resistance or tolerance to basil downy mildew (BDM) race-0 and comprising a haplotype associated with the BDM race-0 resistance or tolerance; and/or selecting a second Ocimum americanum basil plant exhibiting resistance or tolerance to basil downy mildew (BDM) race-1 and comprising a haplotype associated with the BDM race 1 resistance or tolerance; b) crossing the first and/or the second BDM resistant or tolerant basil plant with a basil plant lacking BDM resistance or tolerance, to produce F1 basil progeny plants; c) optionally backcrossing the F1 plants with the BDM nonresistant basil plant; and d) selecting a basil plant having resistance to BDM race-0 and/or BDM race-1, wherein the BDM resistant or tolerant basil plant has introgressed into its genome a haplotype associated with the resistance to BDM race-0 and/or race-1.

It is a further object of the present invention to disclose the method as defined above, wherein said step of crossing comprises steps of: a) crossing the first Ocimum americanum basil plant exhibiting resistance or tolerance to BDM race-0 with a BDM nonresistant Ocimum basilicum basil plant to produce first F1 plants; b) crossing the first Ocimum americanum basil plant exhibiting resistance or tolerance to BDM race-1 with a BDM nonresistant Ocimum basilicum basil plant to produce second F1 plants; c) backcrossing and self-pollinating the first F1 plants to produce first BDM race-0 resistant or tolerant Ocimum basilicum basil plant comprising a haplotype associated with the BDM race-0 resistance or tolerance; d) backcrossing and self-pollinating the second F1 plants to produce second BDM race-resistant or tolerant Ocimum basilicum basil plant comprising a haplotype associated with the BDM race-1 resistance or tolerance; e) crossing the first and second Ocimum basilicum basil plant to produce a double BDM resistance Ocimum basilicum basil plant and/or seed resistant or tolerant to BDM race-0 and BDM race-1.

It is a further object of the present invention to disclose the method as defined in any of the above, wherein the method further comprises a step of analyzing the genomic sequence of the double BDM resistance Ocimum basilicum basil plant and/or seed for the presence of a haplotype associated with the BDM race-0 resistance or tolerance and a haplotype associated with the BDM race-1 resistance or tolerance.

It is a further object of the present invention to disclose the method as defined in any of the above, wherein the double BDM resistance Ocimum basilicum basil plant exhibits synergistic effect with respect to resistance or tolerance to BDM race-0 and/or BDM race-1.

It is a further object of the present invention to disclose the method as defined in any of the above, wherein said BDM resistance or tolerance associated haplotype is derived from Ocimum americanum var. americanum and/or from Ocimum americanum var. pilosum.

It is a further object of the present invention to disclose the method as defined in any of the above, wherein said BDM resistance or tolerance associated haplotype comprises at least one of: a single nucleotide polymorphism (SNP) of nucleotide T at position 7 of a sequence comprising at least 80% identity to SEQ ID NO: 1, SNP of nucleotide A at position 5 of a sequence comprising at least 80% identity to SEQ ID NO:2, SNP of nucleotide C at position 48 of a sequence comprising at least 80% identity to SEQ ID NO:3, SNP of nucleotide G at position 4 of a sequence comprising at least 80% identity to SEQ ID NO:4, SNP of nucleotide T at position 19 of a sequence comprising at least 80% identity to SEQ ID NO:5, SNP of nucleotide A at position 8 of a sequence comprising at least 80% identity to SEQ ID NO:6, SNP of nucleotide T at position 29 of a sequence comprising at least 80% identity to SEQ ID NO:7, SNP of nucleotide T at position 55 of a sequence comprising at least 80% identity to SEQ ID NO:8, SNP of nucleotide T at position 41 of a sequence comprising at least 80% identity to SEQ ID NO:9, SNP of nucleotide C at position 11 of a sequence comprising at least 80% identity to SEQ ID NO: 10, SNP of nucleotide G at position 6 of a sequence comprising at least 80% identity to SEQ ID NO:11 or any combination thereof.

It is a further object of the present invention to disclose the method as defined in any of the above, wherein said BDM resistance or tolerance associated haplotype comprises (a) at least one nucleic acid sequence selected from SEQ ID NO:1, SEQ ID NO:2, SEQ ID NO:3, SEQ ID NO:4, SEQ ID NO: 5, SEQ ID NO:6 and/or SEQ ID NO:7, (b) at least one nucleic acid sequence selected from SEQ ID NO:8, SEQ ID NO:9, SEQ ID NO:10 and/or SEQ ID NO:11 or (c) any combination thereof.

It is a further object of the present invention to disclose the method as defined in any of the above, wherein said SEQ ID NO: 1, SEQ ID NO:2, SEQ ID NO:3, SEQ ID NO:4, SEQ ID NO:5, SEQ ID NO: 6 and/or SEQ ID NO:7 is associated with resistance or tolerance to BDM race-0.

It is a further object of the present invention to disclose the method as defined in any of the above, wherein said SEQ ID NO:8, SEQ ID NO:9, SEQ ID NO: 10 and/or SEQ ID NO:11 is associated with resistance or tolerance to BDM race-1.

It is a further object of the present invention to disclose the method as defined in any of the above, wherein a haplotype comprising a combination of one or more of introgressed SEQ ID NO:1, SEQ ID NO: 2, SEQ ID NO:3, SEQ ID NO:4, SEQ ID NO:5, SEQ ID NO:6 and/or SEQ ID NO:7, together with one or more of SEQ ID NO:8, SEQ ID NO:9, SEQ ID NO: 10 and/or SEQ ID NO:11 is associated with synergistic effect with respect to resistance or tolerance to BDM, specifically to BDM race-0 and BDM race-1, as compared to the effect associated with each of the sequences separately.

It is a further object of the present invention to disclose the method as defined in any of the above, wherein the cultivated basil plant and/or seed is an Ocimum basilicum or any hybrid thereof, having double resistance or tolerance to BDM race-0 and BDM race-1.

It is a further object of the present invention to disclose the method as defined in any of the above, wherein the cultivated basil plant and/or seed is Ocimum basilicum or any hybrid thereof and the BDM resistance or tolerance associated haplotype is derived from Ocimum americanum or any hybrid thereof.

It is a further object of the present invention to disclose the method as defined in any of the above, wherein said cultivated basil plant and/or seed is Ocimum basilicum or any hybrid thereof, and the genome of said plant and/or seed comprises a haplotype comprising at least one of: a single nucleotide polymorphism (SNP) at position 41 of a sequence comprising at least 80% identity to SEQ ID NO: 12, SNP at position 7 of a sequence comprising at least 80% identity to SEQ ID NO:13 and SNP at position 7 of a sequence comprising at least 80% identity to SEQ ID NO:14 or any combination thereof.

It is a further object of the present invention to disclose the method as defined in any of the above, wherein said cultivated basil plant and/or seed is an Ocimum basilicum or any hybrid thereof, and the genome of said plant and/or seed comprises at least one nucleic acid sequence selected from SEQ ID NO:12, SEQ ID NO:13 and SEQ ID NO:14 or any combination thereof.

It is a further object of the present invention to disclose the method as defined in any of the above, wherein the cultivated basil plant and/or seed comprises (a) a haplotype comprising one or more of SEQ ID NO:12-14 or any combination thereof, (b) one or more of SEQ ID NO:1-7 or any combination thereof, and (c) one or more of SEQ ID NO:8-11, or any combination thereof.

It is a further object of the present invention to disclose the method as defined above, wherein the basil plant is Ocimum basilicum exhibiting synergistic effect with respect to resistance or tolerance to BDM race-0 and BDM race-1.

It is a further object of the present invention to disclose the method as defined in any of the above, wherein the cultivated basil plant and/or seed is homozygous or heterozygous to any one of SEQ ID NO: 1-14, or any combination thereof.

It is a further object of the present invention to disclose the method as defined in any of the above, wherein the method comprising: pollinating a BDM nonresistant basil plant with pollen from a wild BDM resistant basil plant; rescuing fertilized ovules from the nonresistant basil plant; growing the rescued fertilized ovules to produce F1 plants; backcrossing the F1 plants with the nonresistant basil plant; and selecting for a basil plant having resistance to BDM, wherein the BDM resistant basil plant has introgressed into its genome the sequence haplotype associated with resistance to BDM.

It is a further object of the present invention to disclose the method as defined above, wherein the rescuing comprises: growing a receptacle separated from a sterile basil plant on MS medium at about 25° C. and then at about 18° C.; transferring immature seed to MS medium to develop plantlets; transfer plantlets to rooting medium; and grow plantlets at about 27° C. to obtain fertile basil plants.

It is a further object of the present invention to disclose a basil plant, plant part or seed produced by the method as defined in any of the above.

It is a further object of the present invention to disclose an allele, haplotype, molecular marker or genetic determinant being inherited to progeny plant, wherein said allele, haplotype, molecular marker or genetic determinant is associated with resistance or tolerance to BDM race-0 or to BDM race-1.

It is a further object of the present invention to disclose the allele, haplotype, molecular marker or genetic determinant as defined above, wherein said allele, haplotype, molecular marker or genetic determinant is selected from: a) a single nucleotide polymorphism (SNP) of nucleotide T at position 7 of a sequence comprising at least 80% identity to SEQ ID NO:1, SNP of nucleotide A at position 5 of a sequence comprising at least 80% identity to SEQ ID NO:2, SNP of nucleotide C at position 48 of a sequence comprising at least 80% identity to SEQ ID NO:3, SNP of nucleotide G at position 4 of a sequence comprising at least 80% identity to SEQ ID NO:4, SNP of nucleotide T at position 19 of a sequence comprising at least 80% identity to SEQ ID NO:5, SNP of nucleotide A at position 8 of a sequence comprising at least 80% identity to SEQ ID NO:6, SNP of nucleotide T at position 29 of a sequence comprising at least 80% identity to SEQ ID NO:7, or any combination thereof, associated with resistance to BDM race-0; b) SNP of nucleotide T at position 55 of a sequence comprising at least 80% identity to SEQ ID NO:8, SNP of nucleotide T at position 41 of a sequence comprising at least 80% identity to SEQ ID NO:9, SNP of nucleotide C at position 11 of a sequence comprising at least 80% identity to SEQ ID NO:10, SNP of nucleotide G at position 6 of a sequence comprising at least 80% identity to SEQ ID NO:11 or any combination thereof, associated with resistance to BDM race-1; and c) any combination thereof.

It is a further object of the present invention to disclose the allele, haplotype, molecular marker or genetic determinant as defined in any of the above, wherein said allele, haplotype, molecular marker or genetic determinant comprising at least one of: a) at least one sequence selected from SEQ ID NO: 1-7, said at least one sequence is associated with resistance or tolerance to BDM race-0; and b) at least one sequence selected from a sequence comprising SEQ ID NO:8-11, said sequence is associated with resistance or tolerance to BDM race-1.

It is a further object of the present invention to disclose an isolated nucleotide sequences annealing with the nucleotide sequence of said allele, haplotype, molecular marker or genetic determinant as defined in any of the above, wherein said sequences are suitable for the detection and/or production of Ocimum basilicum basil plant exhibiting resistance or tolerance to BDM race-0 and/or BDM race-1.

It is a further object of the present invention to disclose the isolated nucleotide sequences as defined above, wherein said sequences suitable for the detection and/or production of Ocimum basilicum basil plant exhibiting resistance or tolerance to BDM race-0 and/or BDM race-1 further comprise (a) one or more of at least one of a single nucleotide polymorphism (SNP) of nucleotide G at position 41 of a sequence comprising at least 80% identity to SEQ ID NO:12, SNP of nucleotide T at position 7 of a sequence comprising at least 80% identity to SEQ ID NO:13 and SNP of nucleotide C at position 7 of a sequence comprising at least 80% identity to SEQ ID NO: 14 or any combination thereof, and/or (b) one or more of SEQ ID NO:12-14, said at least one sequence is associated with Ocimum basilicum basil plant species.

It is a further object of the present invention to disclose an use of an isolated nucleotide sequence selected from at least one of a) a single nucleotide polymorphism (SNP) of nucleotide T at position 7 of a sequence comprising at least 80% identity to SEQ ID NO:1, SNP of nucleotide A at position 5 of a sequence comprising at least 80% identity to SEQ ID NO:2, SNP of nucleotide C at position 48 of a sequence comprising at least 80% identity to SEQ ID NO:3, SNP of nucleotide G at position 4 of a sequence comprising at least 80% identity to SEQ ID NO:4, SNP of nucleotide T at position 19 of a sequence comprising at least 80% identity to SEQ ID NO:5, SNP of nucleotide A at position 8 of a sequence comprising at least 80% identity to SEQ ID NO:6, SNP of nucleotide T at position 29 of a sequence comprising at least 80% identity to SEQ ID NO:7, or any combination thereof, associated with resistance to BDM race-0; b) SNP of nucleotide T at position 55 of a sequence comprising at least 80% identity to SEQ ID NO:8, SNP of nucleotide T at position 41 of a sequence comprising at least 80% identity to SEQ ID NO:9, SNP of nucleotide C at position 11 of a sequence comprising at least 80% identity to SEQ ID NO:10 or SNP of nucleotide G at position 6 of a sequence comprising at least 80% identity to SEQ ID NO:11 or any combination thereof, associated with resistance to BDM race-1; c) at least one of a single nucleotide polymorphism (SNP) of nucleotide G at position 41 of a sequence comprising at least 80% identity to SEQ ID NO: 12, SNP of nucleotide T at position 7 of a sequence comprising at least 80% identity to SEQ ID NO: 13 and SNP of nucleotide C at position 7 of a sequence comprising at least 80% identity to SEQ ID NO:14 or any combination thereof; and d) any combination thereof, for the detection and/or production of Ocimum basilicum basil plant exhibiting resistance or tolerance to BDM race-0 and BDM race-1.

It is a further object of the present invention to disclose the use as defined above, wherein the Ocimum basilicum basil plant exhibit synergistic effect with respect to resistance or tolerance to BDM race-0, BDM race-1.

### BRIEF DESCRIPTION OF THE FIGURES

Exemplary non-limited embodiments of the disclosed subject matter will be described, with reference to the following description of the embodiments, in conjunction with the figures. The figures are generally not shown to scale and any sizes are only meant to be exemplary and not necessarily limiting. Corresponding or like elements are optionally designated by the same numerals or letters.

FIG. 1 is schematically presenting a breeding scheme for obtaining a disease resistant cultivated basil plant according to embodiments of the present invention;

FIG. 2A is schematically presenting a phenotypical screening of leaves performed by visual inspection and classified by the scale of 1-6 according to the symptoms; and

FIG. 2B is a photographic representation of BDM resistance screened leaves classified by the scale 1-6 according to the symptoms.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

Sweet basil ('S. b′ or Ocimum basilicum, 2n=4×=48) is susceptible to downy mildew (DM) caused by Peronospora belbahrii. Pb-1 a gene that was reported to exhibit resistance to the disease, became prone to disease due to occurrence of a new virulent races.

Highly resistant wild Ocimum species are: O. americanum var. pilosum, O. americanum var. americanum (syn canum), O. kilimandscharicum, O. gratissimum, O. campechianum, and O. tenuiflorum. Moderately resistant types are: O. basilicum var. anisatum, O. basilicum var. thyrsiflorum, O. basilicum var. citrodorum, O. xcitrodorum and O. basilicum var. minimum. Commercial cultivars of sweet basils (O. basilicum) are all highly susceptible.

It is within the scope of the present invention that restoration of fertility by substantial backcrossing is absolutely required for successful introgression of resistance genes from wild basil into horticulturally and aromatically improved genetic backgrounds. It is further within the scope that embryo rescue procedures were used to overcome interspecies infertility barrier between Ocimum species. Fertilized F1 ovules were rescued, cultured in vitro, rooted and grown in the greenhouse. BDM resistant BC1 plants showing certain level of fertility were backcrossed several times and eventually became fully fertile.

In the present invention, sources of resistance against Peronospora belbahrii, PI 500945 (O. americanum var. americanum) and PI 500950 (O. americanum var. pilosum), effective against the new emerging race(s) of the pathogen, (BDM race-0 and BDM race-1, respectively), are used to generate a sweet basil (O. basilicum) resistant, or showing synergistic resistance, to both BDM race-0 and BDM race-1. In some embodiments of the present invention the O. basilicum used for the crosses/backcrosses with the source of resistance, encompass within its genome resistance to Fusarium wilt (Fob). In this case a triple resistance O. basilicum would be produced, carrying resistances to BDM race-0, BDM race-1 and Fusarium wilt (Fob), which is highly desirable and was not available until the current invention.

It is emphasized that when these two BDM resistance genes are combined, they act synergistically to suppress the attack of basil leaves with any race of P. belbahrii available so far.

Thus, according to one embodiment, the present invention provides a disease resistant cultivated basil plant and/or seed, wherein said basil plant and/or seed comprising a genomic sequence having an introgressed basil downy mildew (BDM) resistance or tolerance associated haplotype. The aforementioned haplotype is derived from Ocimum americanum or any hybrid thereof.

The present invention is directed to fertile commercially valuable sweet basil plants having an altered genotype comprising introgressed haplotype (from wild basil) associated with resistance or tolerance to BDM (caused by P. belbahrii), and methods for producing the same. The resistance is facilitated by genetic manipulation resulting in introgression of a gene/nucleic acid sequence linked to the resistance or tolerance traits, into the sweet basil plant genome. In certain main aspects, the present invention provides and discloses novel markers combination in sweet basil for a synergistic effect of disease resistances, particularly, BDM race-0 & BDM race-1.

The present invention further provides a method for producing a disease resistant cultivated basil plant and/or seed, the method comprising: (a) selecting a first Ocimum americanum basil plant exhibiting resistance or tolerance to basil downy mildew (BDM) race-0 and comprising a haplotype associated with the BDM race-0 resistance or tolerance; and/or selecting a second Ocimum americanum basil plant exhibiting resistance or tolerance to basil downy mildew (BDM) race-1 and comprising a haplotype associated with the BDM race-1 resistance or tolerance; (b) crossing the first and/or the second BDM resistant or tolerant basil plant with a basil plant lacking BDM resistance or tolerance, to produce F1 basil progeny plants; (c) optionally backcrossing the F1 plants with the BDM nonresistant basil plant; and (d) selecting for a basil plant having resistance to BDM race-0 and/or BDM race-1, wherein the BDM resistant or tolerant basil plant has introgressed into its genome a haplotype associated with the resistance to BDM race-0 and/or race 1.

According to a further aspect of the invention, the step of crossing comprises steps of: (a) crossing the first Ocimum americanum basil plant exhibiting resistance or tolerance to BDM race-0 with a BDM nonresistant Ocimum basilicum basil plant to produce first F1 plants; (b) crossing the first Ocimum americanum basil plant exhibiting resistance or tolerance to BDM race-1 with a BDM nonresistant Ocimum basilicum basil plant to produce second F1 plants; (c) backcrossing and self-pollinating the first F1 plants to produce first BDM race-0 resistant or tolerant Ocimum basilicum basil plant comprising a haplotype associated with the BDM race-0 resistance or tolerance; (d) backcrossing and self-pollinating the second F1 plants to produce second BDM race-1 resistant or tolerant Ocimum basilicum basil plant comprising a haplotype associated with the BDM race-1 resistance or tolerance; and (e) crossing the first and second Ocimum basilicum basil plant to produce a double BDM resistance Ocimum basilicum basil plant and/or seed resistant or tolerant to BDM race-0 and BDM race-1.

As used herein the term “about” denotes ±25% of the defined amount or measure or value.

As used herein the term “similar” denotes a correspondence or resemblance range of about ±20%, particularly ±15%, more particularly about ±10% and even more particularly about ±5%.

As used herein the term “corresponding” generally means similar, analogous, like, alike, akin, parallel, identical, resembling or comparable. In further aspects it means having or participating in the same relationship (such as type or species, kind, degree, position, correspondence, or function). It further means related or accompanying. In some embodiments of the present invention it refers to plants of the same basil species or strain or variety or to sibling plant, or one or more individuals having one or both parents in common or having the same or similar genetic background.

A “plant” as used herein refers to any plant at any stage of development, particularly a seed plant. The term “plant” includes the whole plant or any parts or derivatives thereof, such as plant cells, seeds, plant protoplasts, plant cell tissue culture from which Basil plants can be regenerated, plant callus or calli, meristematic cells, microspores, embryos, immature embryos, pollen, ovules, anthers, fruit, flowers, leaves, cotyledons, pistil, seeds, seed coat, roots, root tips and the like.

The term “plant cell” used herein refers to a structural and physiological unit of a plant, comprising a protoplast and a cell wall. The plant cell may be in a form of an isolated single cell or a cultured cell, or as a part of higher organized unit such as, for example, plant tissue, a plant organ, or a whole plant.

The term “plant cell culture” as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” used herein refers to leaves, stems, roots, root tips, flowers or flower parts, fruits, pollen, egg cells, zygotes, seeds, seed coat, cuttings, cell or tissue cultures, or any other part or product of a plant or a combination thereof.

A “plant organ” as used herein means a distinct and visibly structured and differentiated part of a plant such as a root, stem, leaf, flower, flower bud, or embryo.

The term “plant tissue” as used herein means a group of plant cells organized into a structural and functional unit. Any tissue of a plant in planta or in culture is included. This term includes, but is not limited to, whole plants, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “progeny” or “progenies” refers in a non-limiting manner to offspring or descendant plants. According to certain embodiments, the term “progeny” or “progenies” refers to plants developed or grown or produced from the disclosed or deposited seeds as detailed inter alia. The grown basil plants preferably have the desired traits of the disclosed or deposited seeds, i.e. tolerance or resistance to Basil Downy-Mildew (BDM).

As used herein, the term “basil” or “Ocimum basilicum”, also called great basil, is a culinary herb of the family Lamiaceae (mints). The generic term “basil” includes the variety also known as Sweet basil or Genovese basil. It is an aromatic plant with a spicy odor and flavor. Basil is native to tropical regions from Central Africa to Southeast Asia. In temperate climates basil is treated as an annual plant, however, basil can be grown as a short-lived perennial or biennial in warmer horticultural zones with tropical or Mediterranean climates.

Within the context of the present invention, any basil cultivar, species or variety also include within its scope hybrids of the cultivar, species or variety.

There are many varieties of basil including sweet basil, Thai basil (O. basilicum var. thyrsiflora), and Mrs. Burns' Lemon (O. basilicum var. citriodora). O. basilicum can cross-pollinate with other species of the Ocimum genus, producing hybrids such as lemon basil (O. x citriodorum), African blue basil (O. x kilimandscharicum), O. kilimandscharicum, O. tenuiflorum, O. basilicum, O. basilicum var. anisatum, O. basilicum var. thyrsiflorum, O. basilicum var. citrodorum and O. x citrodorum (Syn O. americanum Lemon Types), O. basilicum var. minimum, and hybrids thereof.

As used herein the term “Basil Downy-Mildew” or “BDM” refers to a devastating foliar disease, nearly exclusive to the basil genus (Ocimum spp.). The disease is caused by the airborne oomycete Peronospora belbahrii. BDM was first described in Uganda in 1933, but reported outside Africa in 2001 (Switzerland), hereafter spread throughout Europe within 6 years, South America & the United States (2007), Israel (2011) and finally Australia (2017). As for today, BDM is considered a pandemic with limited chemical and agrotechnical solutions available for the grower. Resistant sweet basil varieties are commercially available, with variable field-resistance performances. Currently all available varieties are under the risk of a resistance collapse due to the apparent genetic plasticity of the pathogen.

Two races of BDM were described by Ben-Naim et al. (2021) incorporated herein by its entirety. Those races are herein represented as BDM race-0 and BDM race-1. Genetic differences were detected between the two races. The two races of P. belbahrii include: ‘Knafo 3’, collected in Ein-Tamar, and ‘Malawi’ collected from Malawi, Africa. The two races were defined by a bio-assay method using differential plants system.

According to embodiments of the present invention, specific haplotypes were identified associated with each of the resistances, namely BDM race-0 and BDM race-1 derived from wild basil, specifically Ocimum americanum.

As used herein the term “resistance” or “improved resistance” or “tolerance” of a plant to a disease refers to the ability of a plant to restrict the growth and development of a specified pathogen and/or the damage they cause when compared to susceptible plants under similar environmental conditions and pathogen pressure. Resistant plants may exhibit some disease symptoms or damage under pathogen or pest pressure, e.g. fungus, virus, bacterium, whitefly, thrips, spider-mites and nematodes. It may also refer to an indication that the plant is less affected by the disease with respect to yield, survivability and/or other relevant agronomic measures, as compared to a less resistant, more “susceptible” plant. According to some embodiments, resistance is a relative term, indicating that a “resistant” plant survives and/or produces better yields under disease conditions as compared to a different (less resistant) plant. As known in the art, disease “tolerance” is sometimes used interchangeably with disease “resistance.” One of ordinary skill in the art will appreciate that plant resistance to disease conditions varies widely, and can represent a spectrum of more-resistant or less-resistant phenotypes. However, by simple observation and/or assays disclosed herein, one of skill can generally determine the relative resistance or susceptibility of different plants, plant lines or plant families under disease conditions, and furthermore, will also recognize the phenotypic gradations of “resistant”.

As used herein, the term “phenotype” means the detectable characteristics of a cell or organism that can be influenced by gene expression. It may be understood within the scope of the invention to refer to a distinguishable characteristic(s) of a genetically controlled trait.

As used herein, the phrase “phenotypic trait” refers to the appearance or other detectable characteristic of an individual, resulting from the interaction of its genome, proteome and/or metabolome with the environment.

As used herein, the term “genotype” refers to the genetic makeup of an individual cell, cell culture, tissue, organism (e.g., a plant), or group of organisms. In other words it refers to the genetic constitution of a cell or organism. An individual's genotype includes the specific alleles, for one or more genetic marker loci, present in the individual's haplotype. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest. Thus, in some embodiments a genotype comprises a summary of one or more alleles present within an individual at one or more genetic loci. In some embodiments, a genotype is expressed in terms of a haplotype.

The term “haplotype” used herein refers hereinafter generally to a combination of alleles (DNA sequences) at adjacent locations (loci) on a chromosome that are transmitted together. A haplotype may be one locus, several loci, or an entire chromosome depending on the number of recombination events that have occurred between a given set of loci. The term “haplotype” further refers to a set of single-nucleotide polymorphisms (SNPs) on a single chromosome of a chromosome pair that are associated statistically.

As used herein, the term “linkage”, and grammatical variants thereof, refers to the tendency of genes, alleles, sequences and/or genetic markers at different loci on the same chromosome to be associated or to segregate together more often than would be expected by chance, if their transmission were independent, in some embodiments as a consequence of their physical proximity.

As used herein, the term “co-segregate” is understood within the scope of the invention to refer to the tendency for genes, traits and/or genetic markers to segregate or to be inherited together. In a specific embodiment, the presence of a molecular or genetic marker within the genome of the plant is associated with the presence of a trait or a phenotype of the plant, i.e. disease resistance or tolerance phenotype. Alternatively, two or more genes, gene alleles or genetic markers that are linked on the same chromosome are transmitted to the same daughter cell leading to the inheritance by the offspring of these genes or alleles together.

More specifically, in the context of the present invention, the term “co-segregate” refers to the fact that the allele for the trait and the allele(s) for the marker(s) tend to be transmitted together because they are on the same chromosome, and reduced recombination between them resulting in a non-random association of their alleles on the same chromosome. “Co-segregation” also refers to the presence of two or more traits, genetic markers or combinations thereof, within a single plant of which at least one is known to be genetic and which cannot be readily explained by chance. In some embodiments, novel genetic markers are herein identified to co segregate with the BDM resistance trait or phenotype.

The term “co-segregate” used in the present invention is analogous to coupling or co-inheriting in some of the embodiments of the invention.

As used herein, the term “introgression” when used in reference to a genetic locus, refers to introduction of a nucleic acid sequence into a new genetic background, such as through backcrossing. Introgression of a genetic locus can be achieved through plant breeding methods and/or by molecular genetic methods such as, for a non-limiting example, plant transformation techniques and/or methods that provide for homologous recombination, non-homologous recombination, site-specific recombination, and/or genomic modifications that provide for locus substitution or locus conversion.

As used herein, the term “cross”, “crossing”, “cross pollination” or “cross-breeding” refer to the process by which the pollen of one flower on one plant is applied (artificially or naturally) to the stigma (ovule) of a flower on another plant.

The term “backcrossing” used herein is understood within the scope of the invention to refer to a process in which a hybrid progeny is repeatedly crossed back to one of the parents. Such a backcrossing process refers to the repeated crossing of a hybrid progeny back to one of the parental basil plants. The parental basil plant, which contributes the gene for the desired characteristic, is termed the nonrecurrent or donor parent. This terminology refers to the fact that the nonrecurrent parent is used one time in the backcross protocol and therefore does not recur. The parental basil plant to which the gene or genes from the nonrecurrent parent are transferred is known as the recurrent parent as it is used for several rounds in the backcrossing protocol. In a typical backcross protocol, a plant from the original varieties of interest (recurrent parent) is crossed to a plant selected from second varieties (nonrecurrent parent) that carries the single gene of interest to be transferred. The resulting progeny from this cross are then crossed again to the recurrent parent and the process is repeated until a basil plant is obtained wherein essentially all of the desired morphological and physiological characteristics of the recurrent parent are recovered in the converted plant, in addition to the single transferred gene from the nonrecurrent parent. Backcrossing methods can be used with the present invention to improve or introduce a characteristic into the parent lines.

It is also within the scope of the invention that backcrossing refers to a system for incorporating desirable inherited traits into an elite or breeding line. In such a system, the “donor parent” refers to the line containing the gene or trait of interest and the recipient parent or recurrent parent refers to the basil line that is used as the non BDM resistant parent line, which is preferably an elite or breeding plant line that is improved by adding the gene or trait of interest.

As used herein, the phrases “sexually crossed” and “sexual reproduction” in the context of the presently disclosed subject matter refers to the fusion of gametes to produce progeny (e.g., by fertilization, such as to produce seed by pollination in plants). A “sexual cross” or “cross-fertilization” is in some embodiments fertilization of one individual by another (e.g., cross-pollination in plants).

The term “selfing” used herein refers in some embodiments to the production of seed by self-fertilization or self-pollination; i.e., pollen and ovule are from the same plant.

As used herein, the term “locus” (plural: “loci”) refers to any site that has been defined genetically. A locus may be a gene, or part of a gene, or a DNA sequence, and may be occupied by different sequences. A locus may also be defined by a SNP (Single Nucleotide Polymorphism), or by several SNPs. As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein the term “genetic modification” refers hereinafter to genetic manipulation or modulation, which is the direct manipulation of an organism's genes using biotechnology. It also refers to a set of technologies used to change the genetic makeup of cells, including the transfer or introgression of genes within and across species, targeted mutagenesis and genome editing technologies to produce improved organisms.

The term “orthologue” as used herein refers hereinafter to one of two or more homologous gene sequences found in different species.

The term “functional variant” or “functional variant of a nucleic acid or amino acid sequence” as used herein, refers to a variant of a sequence or part of a sequence which retains the biological function of the full non-variant allele and hence has the activity of the expressed gene or protein. A functional variant also comprises a variant of the gene of interest encoding a polypeptide which has sequence alterations that do not affect function of the resulting protein, for example, in non-conserved residues. Also encompassed is a variant that is substantially identical, i.e. has only some sequence variations, for example, in non-conserved residues, to the wild type nucleic acid or amino acid sequences of the alleles as shown herein, and is biologically active.

The term “variety” or “cultivar” used herein means a group of similar plants that by structural features and performance can be identified from other varieties within the same species.

The term “allele” used herein means any of one or more alternative or variant forms of a gene or a genetic unit at a particular locus, all of which alleles relate to one trait or characteristic at a specific locus. In a diploid cell of an organism, alleles of a given gene are located at a specific location, or locus (loci plural) on a chromosome. Alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated with a qualitative trait may comprise alternative or variant forms of various genetic units including those mat are identical or associated with a single gene or multiple genes or their products or even a gene disrupting or controlled by a genetic factor contributing to the phenotype represented by the locus. According to further embodiments, the term “allele” designates any of one or more alternative forms of a gene at a particular locus. Heterozygous alleles are two different alleles at the same locus. Homozygous alleles are two identical alleles at a particular locus. A wild type allele is a naturally occurring allele.

The term “polymorphism” is understood within the scope of the invention to refer to the presence in a population of two or more different forms of a gene, genetic marker, or inherited trait or a gene product obtainable, for example, through alternative splicing, DNA methylation, etc.

As used herein, the term “locus” (loci plural) means a specific place or places or region or a site on a chromosome where for example a gene or genetic marker element or factor is found. In specific embodiments, such a genetic element is contributing to a trait.

As used herein, the term “homozygous” refers to a genetic condition or configuration existing when two identical or like alleles reside at a specific locus, but are positioned individually on corresponding pairs of homologous chromosomes in the cell of a diploid organism.

Conversely, as used herein, the term “heterozygous” means a genetic condition or configuration existing when two different or unlike alleles reside at a specific locus, but are positioned individually on corresponding pairs of homologous chromosomes in the cell of a diploid organism.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

As used herein, the term “germplasm” refers to the totality of the genotypes of a population or other group of individuals (e.g., a species). The term “germplasm” can also refer to plant material; e.g., a group of plants that act as a repository for various alleles. Such germplasm genotypes or populations include plant materials of proven genetic superiority; e.g., for a given environment or geographical area, and plant materials of unknown or unproven genetic value; that are not part of an established breeding population and that do not have a known relationship to a member of the established breeding population.

The terms “hybrid”, “hybrid plant” and “hybrid progeny” used herein refers to an individual produced from genetically different parents (e.g., a genetically heterozygous or mostly heterozygous individual).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereinafter to the amount of characters which match exactly between two different sequences. Hereby, gaps are not counted and the measurement is relational to the shorter of the two sequences.

It is further within the scope that the terms “similarity” and “identity” additionally refer to local homology, identifying domains that are homologous or similar (in nucleotide and/or amino acid sequence). It is acknowledged that bioinformatics tools such as BLAST, SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three-dimensional arrangement of amino acids which may not be adjacent.

As used herein, the terms “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA.

The terms “peptide”, “polypeptide” and “protein” are used interchangeably herein and refer to amino acids in a polymeric form of any length, linked together by peptide bonds.

According to some embodiments, studies were carried out in order to locate potential sources of resistance to basil downy mildew (BDM) among commercial and wild basil species. Varying levels of resistance/susceptibility to BDM caused by Peronospora belbahrii have been reported for different Ocimum species. wild Ocimum species such as O. americanum, O. kilimandscharicum, O. gratissimum, O. campechianum, and O. tenuiflorum showed highly resistant; the close relatives of O. basilicum (O. basilicum var. anisatum, O. basilicum var. thyrsiflorum, O. basilicum var. citrodorum, O. x citrodorum and O. basilicum var. minimum) showed moderately resistant to BDM, while all commercial sweet basil (O. basilicum) cultivars showed highly susceptibility to BDM.

As used herein, the terms “variety” and “cultivar” mean a group of similar plants that by their genetic pedigrees and performance can be identified from other varieties within the same species.

As exemplified in the example section below, hybrids showing high resistance to BDM race-0 and BDM race-1 (e.g., F1 hybrids) may be produced by crossing a plant exhibiting resistance to BDM (e.g., plants of: USDA-Plant Introduction number (‘PI’) 500945, PI 500950, PI 500951 and PI 652053) with a plant exhibiting susceptibility to BDM (e.g., sweet basil), notably those hybrids are sterile.

In some aspects, the term “hybrid” refers to the offspring or progeny of genetically dissimilar plant parents or stock produced as the result of controlled cross-pollination as opposed to a non-hybrid seed produced as the result of natural pollination.

According to further aspects, the term “embryo rescue” refers to the development of viable interspecific hybrids from interspecific crosses, which would normally produce seeds which are aborted. Abortion of embryo is derived from interspecific incompatibility caused by genetic distance of parents or different ploidy. Plant embryos may refer to multicellular structures that have the potential to develop into a new plant. In some other cases, the embryo may be a whole ovary plated on media culture. In other cases, zygotic (embryonic) tissue may be extracted from the ovules (coat) and transferred in to a callus tissue culture.

According to one aspect, the present invention provides a method for producing a sweet basil plant having resistance to BDM. In some embodiments, the produced sweet basil plant is fertile. The method includes the steps of interspecies pollination of BDM nonresistant sweet basil plant, optionally resistant to Fob, with pollen from a wild Ocimum plant resistant to BDM race-0 or BDM race-1; rescuing fertilized ovules from the nonresistant basil plant of each of the crosses; growing the rescued fertilized ovules to F1 plants; backcrossing the F1 plants with the BDM nonresistant basil plant (parental lines) and selecting BDM race-0 or BDM race-1 resistant plants; crossing the resultant BDM race-0 or BDM race-1 resistant plants and selecting for a basil plant having both BDM race-0 and BDM race-1 resistance, optionally together with resistance to Fob.

In one embodiment, the invention provides an edible basil plant (Ocimum spp.) having resistance to downy mildew (BDM race-0 and/or BDM race-1), optionally together with resistance to Fusarium-wilt.

Thus, according to one embodiment, a disease resistant cultivated basil plant and/or seed, wherein said basil plant and/or seed comprising a genomic sequence having an introgressed basil downy mildew (BDM) resistance or tolerance associated haplotype is provided. The haplotype is derived from Ocimum americanum or any hybrid thereof.

According to a further embodiment, the present invention provides the disease resistant cultivated basil plant and/or seed as defined in any of the above, wherein said introgressed BDM resistance or tolerance associated haplotype is derived from Ocimum americanum var. americanum and/or from Ocimum americanum var. pilosum.

According to a further embodiment, the present invention provides the disease resistant cultivated basil plant and/or seed as defined in any of the above, wherein said introgressed BDM resistance or tolerance associated haplotype comprises at least one of: a single nucleotide polymorphism (SNP) of nucleotide T at position 7 of a sequence comprising at least 80% identity to SEQ ID NO: 1, SNP of nucleotide A at position 5 of a sequence comprising at least 80% identity to SEQ ID NO: 2, SNP of nucleotide C at position 48 of a sequence comprising at least 80% identity to SEQ ID NO:3, SNP of nucleotide G at position 4 of a sequence comprising at least 80% identity to SEQ ID NO:4, SNP of nucleotide T at position 19 of a sequence comprising at least 80% identity to SEQ ID NO:5, SNP of nucleotide A at position 8 of a sequence comprising at least 80% identity to SEQ ID NO:6, SNP of nucleotide T at position 29 of a sequence comprising at least 80% identity to SEQ ID NO:7, SNP of nucleotide T at position 55 of a sequence comprising at least 80% identity to SEQ ID NO:8, SNP of nucleotide T at position 41 of a sequence comprising at least 80% identity to SEQ ID NO:9, SNP of nucleotide C at position 11 of a sequence comprising at least 80% identity to SEQ ID NO: 10, SNP of nucleotide G at position 6 of a sequence comprising at least 80% identity to SEQ ID NO:11 or any combination thereof.

According to a further embodiment, the present invention provides the disease resistant cultivated basil plant and/or seed as defined in any of the above, wherein said introgressed BDM resistance or tolerance associated haplotype comprises at least one nucleic acid sequence selected from SEQ ID NO: 1, SEQ ID NO:2, SEQ ID NO:3, SEQ ID NO:4, SEQ ID NO:5, SEQ ID NO:6, SEQ ID NO: 7, SEQ ID NO:8, SEQ ID NO:9, SEQ ID NO: 10, SEQ ID NO:11 or any combination thereof.

According to a further embodiment, the present invention provides the disease resistant cultivated basil plant and/or seed as defined in any of the above, wherein said introgressed BDM resistance or tolerance associated haplotype is associated with conferring resistance or tolerance to BDM race-0, BDM race-1 or a combination thereof.

According to a further embodiment, the present invention provides the disease resistant cultivated basil plant and/or seed as defined in any of the above, wherein said introgressed SEQ ID NO:1, SEQ ID NO:2, SEQ ID NO:3, SEQ ID NO:4, SEQ ID NO:5, SEQ ID NO:6 and/or SEQ ID NO:7 is associated with resistance or tolerance to BDM race-0.

According to a further embodiment, the present invention provides the disease resistant cultivated basil plant and/or seed as defined in any of the above, wherein said introgressed SEQ ID NO:8, SEQ ID NO:9, SEQ ID NO:10 and/or SEQ ID NO:11 is associated with resistance or tolerance to BDM race-1.

According to a further embodiment, the present invention provides the disease resistant cultivated basil plant and/or seed as defined in any of the above, wherein a haplotype comprising a combination of one or more of introgressed SEQ ID NO:1, SEQ ID NO:2, SEQ ID NO:3, SEQ ID NO: 4, SEQ ID NO:5, SEQ ID NO:6 and/or SEQ ID NO:7, together with one or more of SEQ ID NO: 8, SEQ ID NO:9, SEQ ID NO: 10 and/or SEQ ID NO:11 is associated with synergistic effect on resistance or tolerance to BDM, specifically to BDM race-0 and BDM race-1, as compared to the effect associated with each of the sequences separately.

According to a further embodiment, the present invention provides the disease resistant cultivated basil plant and/or seed as defined in any of the above, wherein said basil plant exhibit a synergistic effect with respect to resistance or tolerance to BDM, specifically to BDM race-0 and BDM race-1.

According to a further embodiment, the present invention provides the disease resistant cultivated basil plant and/or seed as defined in any of the above, wherein said resistance or tolerance to BDM is higher relative to a basil plant of the same species lacking the at least one introgressed nucleic acid sequences associated with resistance or tolerance to BDM race-0 or BDM race-1.

According to a further embodiment, the present invention provides the disease resistant cultivated basil plant and/or seed as defined in any of the above, wherein the cultivated basil plant and/or seed is selected from the group consisting of Ocimum kilimandscharicum (O. kilimandscharicum), O. tenuiflorum, O. basilicum, O. basilicum var. anisatum, O. basilicum var. thyrsiflorum, O. basilicum var. citrodorum and O. x citrodorum (Syn O. americanum Lemon Types), O. basilicum var. minimum, Ocimum basilicum var. siam-queen, Ocimum basilicum cv. Perrie, Ocimum basilicum L. and hybrids thereof.

According to a further embodiment, the present invention provides the disease resistant cultivated basil plant and/or seed as defined in any of the above, wherein the cultivated basil plant and/or seed is an Ocimum basilicum plant and/or seed or any hybrid thereof.

According to a further embodiment, the present invention provides the disease resistant cultivated basil plant and/or seed as defined in any of the above, wherein the cultivated basil plant and/or seed is Ocimum basilicum or any hybrid thereof and the introgressed BDM resistance or tolerance associated haplotype is derived from Ocimum americanum or any hybrid thereof.

According to a further embodiment, the present invention provides the disease resistant cultivated basil plant and/or seed as defined in any of the above, wherein said cultivated basil plant and/or seed is Ocimum basilicum or any hybrid thereof, and the genome of said plant and/or seed comprises a haplotype comprising at least one of: a single nucleotide polymorphism (SNP) of nucleotide G at position 41 of a sequence comprising at least 80% identity to SEQ ID NO: 12, SNP of nucleotide T at position 7 of a sequence comprising at least 80% identity to SEQ ID NO: 13 and SNP of nucleotide C at position 7 of a sequence comprising at least 80% identity to SEQ ID NO: 14 or any combination thereof.

According to a further embodiment, the present invention provides the disease resistant cultivated basil plant and/or seed as defined in any of the above, wherein said cultivated basil plant and/or seed is an Ocimum basilicum or any hybrid thereof, and the genome of said plant and/or seed comprises at least one nucleic acid sequence selected from SEQ ID NO: 12, SEQ ID NO:13 and SEQ ID NO:14 or any combination thereof.

According to a further embodiment, the present invention provides the disease resistant cultivated basil plant and/or seed as defined in any of the above, wherein the cultivated basil plant and/or seed comprises a haplotype comprising any one of SEQ ID NO: 12-14 or any combination thereof, together with any one of SEQ ID NO:1-7 or any combination thereof, and any one of SEQ ID NO: 8-11 or any combination thereof.

According to a further embodiment, the present invention provides the disease resistant cultivated basil plant and/or seed as defined in any of the above, wherein the cultivated basil plant and/or seed is an Ocimum basilicum or any hybrid thereof, having double resistance or tolerance to BDM race-0 and BDM race-1.

According to a further embodiment, the present invention provides the disease resistant cultivated basil plant and/or seed as defined in any of the above, wherein said resistant cultivated basil plant is fertile.

According to a further embodiment, the present invention provides the disease resistant cultivated basil plant and/or seed as defined in any of the above, wherein the plant and/or seed is homozygous or heterozygous to any one of SEQ ID NO: 1-14, or any combination thereof.

According to a further embodiment, the present invention provides a disease resistant cultivated basil plant and/or seed, wherein said basil plant and/or seed comprising a genomic sequence having an introgressed basil downy mildew (BDM) resistance or tolerance associated haplotype, wherein said haplotype comprises (a) a single nucleotide polymorphism (SNP) of nucleotide T at position 7 of a sequence comprising at least 80% identity to SEQ ID NO: 1, SNP of nucleotide A at position 5 of a sequence comprising at least 80% identity to SEQ ID NO:2, SNP of nucleotide C at position 48 of a sequence comprising at least 80% identity to SEQ ID NO:3, SNP of nucleotide G at position 4 of a sequence comprising at least 80% identity to SEQ ID NO:4, SNP of nucleotide T at position 19 of a sequence comprising at least 80% identity to SEQ ID NO:5, SNP of nucleotide A at position 8 of a sequence comprising at least 80% identity to SEQ ID NO:6, SNP of nucleotide T at position 29 of a sequence comprising at least 80% identity to SEQ ID NO:7, or any combination thereof, associated with resistance to BDM race-0; and SNP of nucleotide T at position 55 of a sequence comprising at least 80% identity to SEQ ID NO:8, SNP of nucleotide T at position 41 of a sequence comprising at least 80% identity to SEQ ID NO:9, SNP of nucleotide Cat position 11 of a sequence comprising at least 80% identity to SEQ ID NO: 10, SNP of nucleotide G at position 6 of a sequence comprising at least 80% identity to SEQ ID NO:11 or any combination thereof, associated with resistance to BDM race-1; and wherein said cultivated basil plant exhibits synergistic effect with respect to BDM resistance, as compared to the effect exhibited by a basil plant comprising only one of BDM race-0 or BDM race-1 associated haplotype.

According to a further embodiment, the present invention provides a plant part comprising at least one regenerable cell, pollen, ovule, fruit or seed of the disease resistant cultivated basil plant as defined in any of the above.

According to a further embodiment, the present invention provides a tissue culture of regenerable cells, protoplasts or callus obtained from the disease resistant cultivated basil plant as defined in any of the above.

It is further within the scope of the present invention to provide a method for producing a disease resistant cultivated basil plant and/or seed, the method comprising: (a) selecting a first Ocimum americanum basil plant exhibiting resistance or tolerance to basil downy mildew (BDM) race-0 and comprising a haplotype associated with the BDM race-0 resistance or tolerance; and/or selecting a second Ocimum americanum basil plant exhibiting resistance or tolerance to basil downy mildew (BDM) race-1 and comprising a haplotype associated with the BDM race-1 resistance or tolerance; (b) crossing the first and/or the second BDM resistant or tolerant basil plant with a basil plant lacking BDM resistance or tolerance, to produce F1 basil progeny plants; (c) optionally backcrossing the F1 plants with the BDM nonresistant basil plant; and (d) selecting for a basil plant having resistance to BDM race-0 and/or BDM race-1, wherein the BDM resistant or tolerant basil plant has introgressed into its genome a haplotype associated with the resistance to BDM race-0 and/or race-1.

It is further within the scope of the present invention to provide the method as defined in any of the above, wherein said step of crossing comprises steps of: (a) crossing the first Ocimum americanum basil plant exhibiting resistance or tolerance to BDM race-0 with a BDM nonresistant Ocimum basilicum basil plant to produce first F1 plants; (b) crossing the first Ocimum americanum basil plant exhibiting resistance or tolerance to BDM race-1 with a BDM nonresistant Ocimum basilicum basil plant to produce second F1 plants; (c) backcrossing and self-pollinating the first F1 plants to produce first BDM race-0 resistant or tolerant Ocimum basilicum basil plant comprising a haplotype associated with the BDM race-0 resistance or tolerance; (d) backcrossing and self-pollinating the second F1 plants to produce second BDM race-1 resistant or tolerant Ocimum basilicum basil plant comprising a haplotype associated with the BDM race-1 resistance or tolerance; (e) crossing the first and second Ocimum basilicum basil plant to produce a double BDM resistance Ocimum basilicum basil plant and/or seed resistant or tolerant to BDM race-0 and BDM race-1.

It is further within the scope of the present invention to provide the method as defined in any of the above, wherein the method further comprises a step of analyzing the genomic sequence of the double BDM resistance Ocimum basilicum basil plant and/or seed for the presence of a haplotype associated with the BDM race-0 resistance or tolerance and a haplotype associated with the BDM race-1 resistance or tolerance.

It is further within the scope of the present invention to provide the method as defined in any of the above, wherein the double BDM resistance Ocimum basilicum basil plant exhibits synergistic effect with respect to resistance or tolerance to BDM race-0 and/or BDM race-1.

It is further within the scope of the present invention to provide the method as defined in any of the above, wherein said BDM resistance or tolerance associated haplotype is derived from Ocimum americanum var. americanum and/or from Ocimum americanum var. pilosum.

It is further within the scope of the present invention to provide the method as defined in any of the above, wherein said BDM resistance or tolerance associated haplotype comprises at least one of: a single nucleotide polymorphism (SNP) of nucleotide T at position 7 of a sequence comprising at least 80% identity to SEQ ID NO:1, SNP of nucleotide A at position 5 of a sequence comprising at least 80% identity to SEQ ID NO:2, SNP of nucleotide C at position 48 of a sequence comprising at least 80% identity to SEQ ID NO:3, SNP of nucleotide G at position 4 of a sequence comprising at least 80% identity to SEQ ID NO:4, SNP of nucleotide T at position 19 of a sequence comprising at least 80% identity to SEQ ID NO:5, SNP of nucleotide A at position 8 of a sequence comprising at least 80% identity to SEQ ID NO:6, SNP of nucleotide T at position 29 of a sequence comprising at least 80% identity to SEQ ID NO:7, SNP of nucleotide T at position 55 of a sequence comprising at least 80% identity to SEQ ID NO:8, SNP of nucleotide T at position 41 of a sequence comprising at least 80% identity to SEQ ID NO:9, SNP of nucleotide C at position 11 of a sequence comprising at least 80% identity to SEQ ID NO: 10, SNP of nucleotide G at position 6 of a sequence comprising at least 80% identity to SEQ ID NO:11 or any combination thereof.

It is further within the scope of the present invention to provide the method as defined in any of the above, wherein said BDM resistance or tolerance associated haplotype comprises (a) at least one nucleic acid sequence selected from SEQ ID NO:1, SEQ ID NO:2, SEQ ID NO:3, SEQ ID NO: 4, SEQ ID NO:5, SEQ ID NO:6 and/or SEQ ID NO:7, (b) at least one nucleic acid sequence selected from SEQ ID NO:8, SEQ ID NO:9, SEQ ID NO: 10 and/or SEQ ID NO:11 or (c) any combination thereof.

It is further within the scope of the present invention to provide the method as defined in any of the above, wherein said SEQ ID NO:1, SEQ ID NO:2, SEQ ID NO:3, SEQ ID NO:4, SEQ ID NO: 5, SEQ ID NO:6 and/or SEQ ID NO:7 is associated with resistance or tolerance to BDM race-0.

It is further within the scope of the present invention to provide the method as defined in any of the above, wherein said SEQ ID NO:8, SEQ ID NO:9, SEQ ID NO: 10 and/or SEQ ID NO: 11 is associated with resistance or tolerance to BDM race-1.

It is further within the scope of the present invention to provide the method as defined in any of the above, wherein a haplotype comprising a combination of one or more of introgressed SEQ ID SEQ ID NO:1, SEQ ID NO:2, SEQ ID NO:3, SEQ ID NO:4, SEQ ID NO:5, SEQ ID NO:6 and/or SEQ ID NO:7, together with one or more of SEQ ID NO:8, SEQ ID NO:9, SEQ ID NO: 10 and/or SEQ ID NO:11 is associated with synergistic effect with respect to resistance or tolerance to BDM, specifically to BDM race-0 and BDM race-1, as compared to the effect associated with each of the sequences separately.

It is further within the scope of the present invention to provide the method as defined in any of the above, wherein the cultivated basil plant and/or seed is an Ocimum basilicum or any hybrid thereof, having double resistance or tolerance to BDM race-0 and BDM race-1.

It is further within the scope of the present invention to provide the method as defined in any of the above, wherein the cultivated basil plant and/or seed is Ocimum basilicum or any hybrid thereof and the BDM resistance or tolerance associated haplotype is derived from Ocimum americanum or any hybrid thereof.

It is further within the scope of the present invention to provide the method as defined in any of the above, wherein said cultivated basil plant and/or seed is Ocimum basilicum or any hybrid thereof, and the genome of said plant and/or seed comprises a haplotype comprising at least one of: a single nucleotide polymorphism (SNP) at position 41 of a sequence comprising at least 80% identity to SEQ ID NO:12, SNP at position 7 of a sequence comprising at least 80% identity to SEQ ID NO:13 and SNP at position 7 of a sequence comprising at least 80% identity to SEQ ID NO: 14 or any combination thereof.

It is further within the scope of the present invention to provide the method as defined in any of the above, wherein said cultivated basil plant and/or seed is an Ocimum basilicum or any hybrid thereof, and the genome of said plant and/or seed comprises at least one nucleic acid sequence selected from SEQ ID NO: 12, SEQ ID NO:13 and SEQ ID NO: 14 or any combination thereof.

It is further within the scope of the present invention to provide the method as defined in any of the above, wherein the cultivated basil plant and/or seed comprises a haplotype comprising any one of SEQ ID NO:12-14 or any combination thereof, together with any one of SEQ ID NO: 1-7 or any combination thereof, and any one of SEQ ID NO:8-11, or any combination thereof.

It is further within the scope of the present invention to provide the method as defined in any of the above, wherein the cultivated basil plant and/or seed is homozygous or heterozygous to any one of SEQ ID NO:1-14, or any combination thereof.

It is further within the scope of the present invention to provide the method as defined in any of the above, wherein the method comprising: pollinating a BDM nonresistant basil plant with pollen from a wild BDM resistant basil plant; rescuing fertilized ovules from the nonresistant basil plant; growing the rescued fertilized ovules to produce F1 plants; backcrossing the F1 plants with the nonresistant basil plant; and selecting for a basil plant having resistance to BDM, wherein the BDM resistant basil plant has introgressed into its genome the sequence haplotype associated with resistance to BDM.

It is further within the scope of the present invention to provide the method as defined in any of the above, wherein the rescuing comprises: growing a receptacle separated from a sterile basil plant on MS medium at about 25° C. and then at about 18° C.; transferring immature seed to MS medium to develop plantlets; transfer plantlets to rooting medium; and grow plantlets at about 27° C. to obtain fertile basil plants.

It is further within the scope of the present invention to provide a basil plant, plant part or seed produced by the method as defined in any of the above.

According to a further embodiment, the present invention provides an allele, haplotype, molecular marker or genetic determinant being inherited to progeny plant, wherein said allele, haplotype, molecular marker or genetic determinant is associated with resistance or tolerance to BDM race-0 or to BDM race-1.

According to a further embodiment, the present invention provides the allele, haplotype, molecular marker or genetic determinant as defined in any of the above, wherein said allele, haplotype, molecular marker or genetic determinant is selected from: (a) a single nucleotide polymorphism (SNP) of nucleotide T at position 7 of a sequence comprising at least 80% identity to SEQ ID NO: 1, SNP of nucleotide A at position 5 of a sequence comprising at least 80% identity to SEQ ID NO: 2, SNP of nucleotide C at position 48 of a sequence comprising at least 80% identity to SEQ ID NO:3, SNP of nucleotide G at position 4 of a sequence comprising at least 80% identity to SEQ ID NO:4, SNP of nucleotide T at position 19 of a sequence comprising at least 80% identity to SEQ ID NO:5, SNP of nucleotide A at position 8 of a sequence comprising at least 80% identity to SEQ ID NO:6, SNP of nucleotide T at position 29 of a sequence comprising at least 80% identity to SEQ ID NO:7, or any combination thereof, associated with resistance to BDM race-0; (b) SNP of nucleotide T at position 55 of a sequence comprising at least 80% identity to SEQ ID NO:8, SNP of nucleotide T at position 41 of a sequence comprising at least 80% identity to SEQ ID NO: 9, SNP of nucleotide C at position 11 of a sequence comprising at least 80% identity to SEQ ID NO: 10, SNP of nucleotide G at position 6 of a sequence comprising at least 80% identity to SEQ ID NO: 11 or any combination thereof, associated with resistance to BDM race-1; and (c) any combination thereof.

According to a further embodiment, the present invention provides the allele, haplotype, molecular marker or genetic determinant as defined in any of the above, wherein said allele, haplotype, molecular marker or genetic determinant comprising at least one of: (a) at least one sequence selected from SEQ ID NO: 1-7, said at least one sequence is associated with resistance or tolerance to BDM race-0; (b) at least one sequence selected from a sequence comprising SEQ ID NO:8-11, said sequence is associated with resistance or tolerance to BDM race-1.

According to a further embodiment, the present invention provides isolated nucleotide sequences annealing with the nucleotide sequence of said allele, haplotype, molecular marker or genetic determinant as defined in any of the above, wherein said sequences are suitable for the detection and/or production of Ocimum basilicum basil plant exhibiting resistance or tolerance to BDM race-0 and/or BDM race-1.

According to a further embodiment, the present invention provides the isolated nucleotide sequences as defined above, wherein said sequences suitable for the detection and/or production of Ocimum basilicum basil plant exhibiting resistance or tolerance to BDM race-0 and/or BDM race-1 further comprise (a) one or more of at least one of a single nucleotide polymorphism (SNP) of nucleotide G at position 41 of a sequence comprising at least 80% identity to SEQ ID NO: 12, SNP of nucleotide T at position 7 of a sequence comprising at least 80% identity to SEQ ID NO: 13 and SNP of nucleotide C at position 7 of a sequence comprising at least 80% identity to SEQ ID NO: 14 or any combination thereof, and/or (b) one or more of SEQ ID NO: 12-14, said at least one sequence is associated with Ocimum basilicum basil plant species.

According to a further embodiment, the present invention provides use of an isolated nucleotide sequence selected from at least one of (a) at least one of a single nucleotide polymorphism (SNP) of nucleotide G at position 41 of a sequence comprising at least 80% identity to SEQ ID NO:12, SNP of nucleotide T at position 7 of a sequence comprising at least 80% identity to SEQ ID NO: 13 and SNP of nucleotide C at position 7 of a sequence comprising at least 80% identity to SEQ ID NO: 14 or any combination thereof; (b) a single nucleotide polymorphism (SNP) of nucleotide T at position 7 of a sequence comprising at least 80% identity to SEQ ID NO:1, SNP of nucleotide A at position 5 of a sequence comprising at least 80% identity to SEQ ID NO:2, SNP of nucleotide C at position 48 of a sequence comprising at least 80% identity to SEQ ID NO:3, SNP of nucleotide G at position 4 of a sequence comprising at least 80% identity to SEQ ID NO:4, SNP of nucleotide T at position 19 of a sequence comprising at least 80% identity to SEQ ID NO:5, SNP of nucleotide A at position 8 of a sequence comprising at least 80% identity to SEQ ID NO:6, SNP of nucleotide T at position 29 of a sequence comprising at least 80% identity to SEQ ID NO:7, or any combination thereof, associated with resistance to BDM race-0; (c) SNP of nucleotide T at position 55 of a sequence comprising at least 80% identity to SEQ ID NO:8, SNP of nucleotide T at position 41 of a sequence comprising at least 80% identity to SEQ ID NO:9, SNP of nucleotide C at position 11 of a sequence comprising at least 80% identity to SEQ ID NO:10, SNP of nucleotide G at position 6 of a sequence comprising at least 80% identity to SEQ ID NO:11 or any combination thereof, associated with resistance to BDM race-1; and (d) any combination thereof, for the detection and/or production of Ocimum basilicum basil plant exhibiting resistance or tolerance to BDM race-0 and BDM race-1.

According to a further embodiment, the present invention provides the use as defined in any of the above, wherein the Ocimum basilicum basil plant exhibit synergistic effect with respect to resistance or tolerance to BDM race-0, BDM race-1.

In order to understand the invention and to see how it may be implemented in practice, a plurality of preferred embodiments will now be described, by way of non-limiting example only, with reference to the following examples.

### Example 1

### Production of a Double Resistance BDM Race-0/BDM Race-1 Basil Plant

### Materials & Methods

### Downy-Mildew (BDM)

### Directed Pathogen Inoculation Assay

Peronospora belbahrii is a plant pathogen which is the cause of Downy-mildew in basil (BDM). Two races of BDM were identified: BDM race-0 (collected in Ein-Tamar Israel, 2017) and BDM race-1 (collected from Ashalim Israel, 2019). BDM race-0 was maintained by repeated inoculations of ‘Sweet basil’ (a cultivar with no BDM resistance) whereas BDM race-1 was maintained by repeated inoculations of the basil line ‘12-4-6’ (carrying the unknown Pb-1 resistance gene).

Fresh BDM spores were extracted from sporulating plants into cold distilled water. Spores suspension was adjusted to ˜5000 spores/ml and inoculated by spraying onto the upper leaf surfaces (16 days old plants at first 2-4 leaf stage), with the aid of a fine glass atomizer. Inoculated plants were incubated in a dark dew chamber (18° C., 15 h) to ensure infection, and thereafter, under continuous illumination to allow symptoms development (6 days, 25° C.). Plants were returned to the dew chamber on the 7^{th} day post inoculation to enable sporulation on leaves.

### Phenotype Screening

About 8-14 days post inoculation, plants were screened for disease symptoms. Both BDM races show similar symptoms of yellow lesions on upper leaf and dark spores under leaf.

### BDM Phenotypic Scoring

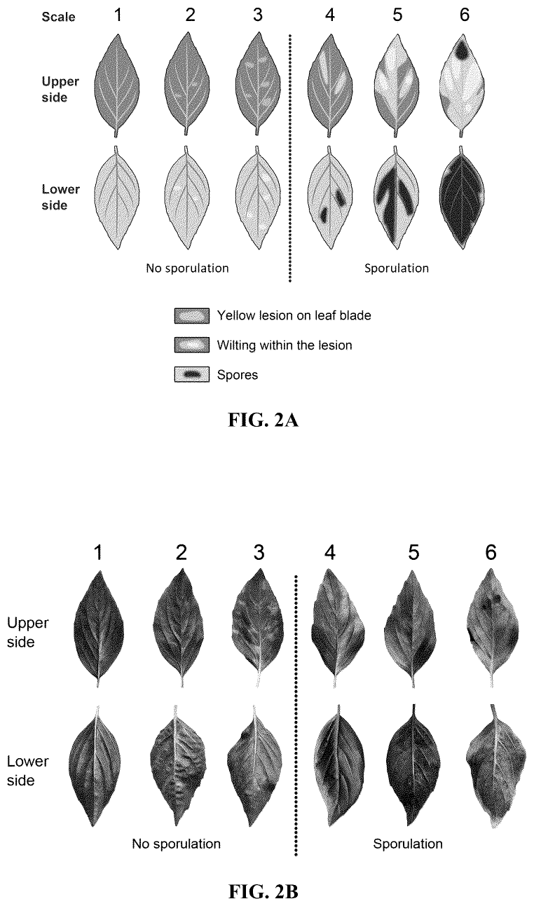

Plants were phenotypically screened and scored by a visual inspection of the leaves a few times during a 2 weeks period. A scale of 1-6 was defined as follows (FIG. 2A & FIG. 2B):
1=no symptoms.
2=1 to 4 fade yellow lesions on leaf blade; no sporulation.
3=>5 yellow lesions; no sporulation.
4=yellow lesions on upper leaf & dark spores on lower leaf; both cover <20% of leaf surface.
5=yellow lesions on upper leaf & dark spores on lower leaf; cover 20-50% of leaf surface.
6=yellow lesions on upper leaf & dark spores on lower leaf; cover >50% of leaf surface. Spores may also appear on upper leaf.

### Genotyping by Sequencing Assay

### Mapping Population

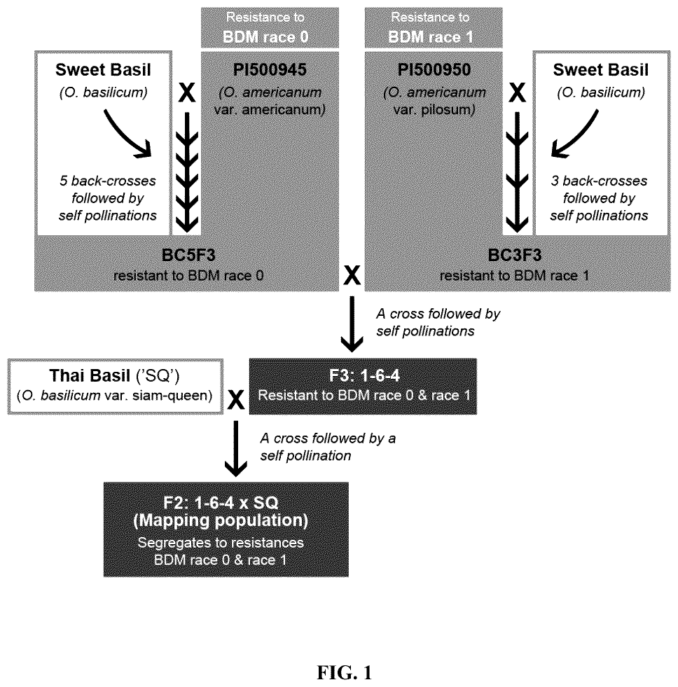

In order to identify markers that are related to BDM resistance, we have created a mapping population that segregates to the following 2 resistance traits: BDM race-0 & BDM race-1. The mapping population was obtained as described below (see also breeding scheme of FIG. 1):

### 1) Obtaining a BDM Race-0 Resistant Line

Resistance source is PI500945 (Ocimum americanum var. americanum, USDA). Although showed a strong incompatibility, the line was successfully crossed with a BDM susceptible ‘sweet basil’ (Ocimum basilicum) to create a fertile progeny (Ben-Naim et al., 2017 “Transfer of Downy Mildew Resistance from Wild Basil (Ocimum americanum) to Sweet Basil (O. basilicum)”, incorporated herein by its entirety). After 5 back-crosses with ‘sweet basil’, a line which presents features of a sweet basil as well as a resistance to BDM race-0 was obtained (line ‘BC5F3’). Resistance to BDM race-0 is controlled by an unknown dominant gene (‘Pb-1’ resistance gene).

### 2) Obtaining a BDM Race-1 Resistant Line

Resistance source is PI500950 (Ocimum americanum var. pilosum, USDA). This line also showed cross-incompatibility with the sweet basil, an obstacle that was overcame by similar means to create a fertile progeny with a sweet basil (Ben-Naim, 2021). After 3 back-crosses with sweet basil a line which presents features of sweet basil as well as the resistance to BDM race-1 (line ‘BC3F3’) was obtained. The BDM race-1 resistance is also controlled by an unknown dominant gene (‘Pb-2’ resistance gene).

### 3) Obtaining a Double Resistance Line, BDM Race-0/BDM Race-1

A cross between BC5F3 and BC3F3 and selection of plants fixed to 2 races of BDM, obtained a line (‘1-6-4’) which is resistant to BDM race-0 and BDM race-1.

### 4) Obtaining a Segregating Population to 2 Resistance Phenotypes (BDM Race-0 & BDM Race-1)

Crossing of ‘1-6-4’ with the susceptible ‘SQ’ variety (Ocimum basilicum′ var. siam-queen) enabled to obtain an F2 population which segregates to the above 2 resistance phenotypes. 239 plants of this population were directly inoculated separately by each fungus, phenotyped and plant tissue was collected in order to be used as a mapping population for the DArTseq assay.

### Targeted Genotyping Assay

### Tissue Collection

Apical leaves from each of the 239 plants and the parental lines of the population were collected into liquid nitrogen. 10-15 mg of lyophilize-dried tissue was sent to DArT (Diversity arrays technology ltd., Australia), for targeted genotyping assay.

### DArTseq Assay

Diversity Arrays Technology (DArT) is a sequence-independent genotyping method that generates genome-wide genetic fingerprints. It enables a high-throughput genotyping of basil regardless of low availability of previous genomic resources.

Genomic DNA is digested with restriction enzymes (PstI and TaqI), adapters are ligated to PstI ends followed by amplification of the adapter-ligated fragments. The amplicon is decoded through hybridization to a microarray which contains representation ˜60,000 fragments (probes) that cover the gene pool of basil. Hybridization of a reference DNA to the array enables quantification of each probe, by fluorescence detection of the amount of DNA spotted on the array during routine assays (analyzed by DArTsoft software). Gathered information is managed by DArTbd, laboratory information management system. A marker is considered polymorphic if the relative hybridization intensity across genotypes falls into distinct clusters. Depending on the cluster into which it falls a marker is called ‘present’ or ‘absent’ (0 or 1) for a particular genotype in a dominant fashion. The accuracy of the assay is approximately 99.8%. The genomic profiling of each individual plant allowed phenotype-genotype association study and the detection of the markers that are most closely linked to Pb-1 and Pb-2 resistance genes (see www.diversityarrays.com/technology-and-resources/targeted-genotyping/)

### Results

Novel genetic markers were identified as detailed in Table 1.

TABLE 1
Identified molecular markers of the present invention

| | SEQ | | | Chrom. | |
|---|---|---|---|---|---|
| | ID | SNP | | (Ocimum | Chromosome |
| Title | No. | polymorphism | Sequence | basilicum 0.1) | Position |
| BDM | 1 | 7: T > A | GACATTTTGCAAACTGG | Scaffold 64 | 9,880,756 |
| race-0 | | | AAAAACGATTTTCATCA | | |
| markers | | | GCTCAACTTTACAGATC | | |
| | | | GGAAGAGCGGTTC | | |
| | 2 | 5: A > G | GTCTATCCAGAAGCAGA | Scaffold 113 | 1,383,495 |
| | | | ATGAGGCGATCACCGG | | |
| | | | GAAGATTTTCTTGCTGG | | |
| | | | ACGATCTCCCTCTT | | |
| | 3 | 48: C > A | AAAACGGAATCTAGGG | Scaffold 113 | 3,907,479 |
| | | | TTTTCAGCACTTCTTTTG | | |
| | | | CTAGTTTGGGTGACGAA | | |
| | | | ACAACGATTACAG | | |
| | 4 | 4: G > A | AAGGTGGAATCTAGGG | Scaffold 113 | 4,073,438 |
| | | | TTTTGAGCACTTCTTTC | | |
| | | | GCGAGTTCGGGGGAAG | | |
| | | | AAATGACGATTACAG | | |
| | 5 | 19: T > G | ATGAAGCGGACTTGGAT | Scaffold 113 | 7,087,081 |
| | | | CCTCACGTCGGTGGTGA | | |
| | | | GTTTATTCTATTTACCGT | | |
| | | | ATATGAACAGTT | | |
| | 6 | 8: A > T | TTGAATAATCATTTTCT | Scaffold462 | 9,173,602 |
| | | | TTCCAAAATTGTTGAGC | | |
| | | | AGTTGGCTGCATAGTCA | | |
| | | | ATTAC | | |
| | 7 | 29: T > G | CATTAGTCCCCGAAGCT | Scaffold15339 | 656,913 |
| | | | CCGGATGTGAATATATG | | |
| | | | GTTTTTCTGGAAAGAAA | | |
| | | | GCGATCGAAAATC | | |
| BDM | 8 | 55: T > C | ATAGCTGAACCCCATCG | Scaffold4377 | 1,090,578 |
| race-1 | | | ACAACACCATCTGCATG | | |
| markers | | | CTGCTGCTCAGAACTAT | | |
| | | | CGGTCCCAGCTCC | | |
| | 9 | 41: T > A | TAGCCGCTGCCGCTTGA | Scaffold4377 | 1,750,273 |
| | | | GAAGCTGCATTAGTTGT | | |
| | | | TTCAGCTGCAACTAAAT | | |
| | | | CTTCAGCAACCAG | | |
| | 10 | 11: C > T | TAGAAGGAGACCCATA | Scaffold4377 | 1,751,698 |
| | | | TTCATCAAAAAGTCGCA | | |
| | | | TAAGATCTCCAAACTGG | | |
| | | | CCGTGCAAATCACC | | |
| | 11 | 6: G > T | CAGCAGGAGAAGGTGT | Scaffold112 | 478,475 |
| | | | ATGGTGGTGAACAGAG | | |
| | | | TACGAGTGATCCCTCTT | | |
| | | | CATCAAATATATCTA | | |
| Reference | 12 | 41: G > A | AAAGAGAAAAGGTTAT | scaffold4335 | 4,906,416 |
| genome | | | GGGCGACCCAAAGTCC | | |
| markers | | | ATAATTCCGGCAGCATT | | |
| | | | TGTTTCATTCAAAAG | | |
| | 13 | 7: T > A | AAAAAATAAATAAATA | scaffold310 | 11,794,989 |
| | | | AATCCAGATAAGAACA | | |
| | | | AATAAACTAATTGTAGA | | |
| | | | GCGGTGAGCAAGGTC | | |
| | 14 | 7: C > G | AAATGGCCAAGTCCTGC | Scaffold14686 | 17,472,254 |
| | | | AAAGGCCTGGCTATGG | | |
| | | | AGCTCGTCAAATGTCTC | | |
| | | | AGTGAGTCCGACTG | | |

As presented in Table 1, molecular markers identifying the Basil plants of the present invention were found by the breeding scheme and assays described above. These genetic markers include specific SNPs associated with the desirable characteristics of:
1) SEQ ID NO: 1-7: resistance against basil downy-mildew race-0 (BDM race-0)
2) SEQ ID NO: 8-11: resistance against basil downy-mildew race-1 (BDM race-1)
1) SEQ ID NO: 12-14: commercial sweet basil Reference Genome (The genome sequence of tetraploid sweet basil, Ocimum basilicum L., provides tools for advanced genome editing and molecular breeding (2020). Itay Gonda, Adi Faigenboim, Chen Adler, Renana Milavski, Merrie-Jean Karp, Alona Shachter, Gil Ronen, Kobi Baruch, David Chaimovitsh, Nativ Dudai. DNA Research, Volume 27, Issue 5, incorporated herein by its entirety):
   Ocimum basilicum cv. Perrie (v0.1, id59011):
Genome ID: 59011
Organism: Ocimum basilicum cv. Perrie
Version: 0.1
Type: scaffolds
Source: Unit of Aromatic and Medicinal Plants
Link: academic.oup.com/dnaresearch/article/27/5/dsaa027/6042144?login=true

Thus, Table 1 presents DNA fragments containing specific SNPs identified as being in association with sweet basil Ocimum basilicum (SEQ ID NO: 12-14) or with the traits of resistance or tolerance to basil downy-mildew race-0 (SEQ ID NO: 1-7) or race-1 (SEQ ID NO: 8-11).

Reference is now made to SEQ ID NO: 12-14, these sequences encompass SNPs that are associated with Ocimum basilicum genome and are appear differently in the wild basil species O. americanum, which is the genetic source of resistance to both BDM race-0 & BDM race-1. Those SNPs are expected to appear at a homozygous allelic state (GG, TT & CC, respectively).

Reference is now made to SEQ ID NO:1-7 and SEQ ID NO:8-11, these sequences encompass SNPs associated with resistance to BDM race-0 and BDM race-1, respectively.

All BDM SNPs (SEQ ID NOs: 1-11) acquire resistance in a dominant manner (meaning both homozygous and heterozygous allelic states are associated with resistance). It is emphasized that the combination between these SNP-associated traits provides a synergistic effect with respect to overall resistance against basil downy mildew, including against BDM race-0 and BDM race-1. This means that the combination of one or more of sequences SEQ ID NO:1-7, together with one or more of SEQ ID NO:8-11, is linked to a higher level of resistance against any one of BDM race-0 and BDM race-1. This synergistic resistance effect would not appear in case of a plant which possess only one or more sequence from SEQ ID NO: 1-7 or one or more sequence from SEQ ID NO: 8-11, but not a combination of the two (at least one sequence of SEQ ID NO:1-7 and at least one sequence of SEQ ID NO:8-11) (see Table 2).

Reference is now made to Table 2, presenting allelic states of representative SNPs, one of each of the BDM races resistance markers (SEQ ID NO:7 for BDM-0 & SEQ ID NO:8 for BDM-1) and their consequential phenotypes. Phenotypic scoring legend appears in the description of FIG. 2. In the table, resistant plants (scores 1, 2 & 3) are presented in bold-italic format.

TABLE 2
Allelic states of representative SNPs, one of each of the
BDM races resistance markers (SEQ ID NO: 7 for BDM-0 & SEQ
ID NO: 8 for BDM-1) and their consequential phenotypes

SEQ ID NO: 7 allelic
state (BDM-0)

| | |
|---|---|
| TT or TG | GG |

A

| | | | |
|---|---|---|---|
| SEQ ID NO: 8 allelic | TT or TC | 1 | 3-5 |
| state (BDM-1) | CC | 1-2 | 5-6 |

B

| | | | |
|---|---|---|---|
| SEQ ID NO: 8 allelic | TT or TC | 1 | 1-3 |
| state (BDM-1) | CC | 5-6 | 5-6 |

### In Table 2:

Part A. phenotypic scoring of plants inoculated with BDM race-0; at least one resistance allele (nucleotide T in position 29 of SEQ ID NO: 7) acquire a resistance to BDM-0 regardless of the allelic state of SEQ ID NO: 8.
Part B. phenotypic scoring of plants inoculated with BDM race-1; at least one resistance allele (nucleotide T in position 55 of SEQ ID NO: 8) acquire a resistance to BDM-1 regardless of the allelic state of SEQ ID NO: 7.

Combining the results from both Table 2 parts (A & B) shows that only a double resistant state to both markers (SEQ ID NO: 7 & SEQ ID NO: 8) acquires a wide resistance to the two tested BDM races (a cell with underlined text).

Thus, the novel molecular markers of the present invention generate a unique haplotype which enable the production and identification of commercially valuable Ocimum basilicum (sweet basil) plants characterized by the desirable combination of phenotypic traits of enhanced resistance to both BDM race-0 & BDM race-1. According to embodiments of the present invention, the unique haplotype comprises a combination of (1) one or more of SEQ ID NO: 12-14, (2) one or more of SEQ ID NO:1-7, and (3) one or more of SEQ ID NO:8-11. It is strongly emphasized that such a basil plant and combination of genetic markers was not known or available prior to the current invention.

Therefore, it is within the scope of the present invention that commercially valuable Ocimum basilicum (sweet basil) plants comprising double resistance to BDM race-0 and BDM race-1 are obtained and disclosed herein for the first time.

It is evident by the results disclosed herein that the present invention provides novel and unique combination of molecular markers surprisingly useful in identifying and providing O. basilicum basil plants with enhanced or synergistic resistance to basil downy-mildew (BDM) races, including BDM race-0 and BDM race-1. Such basil plants are highly commercially valuable.

### REFERENCES

Ben-Naim Y, Falach L, Cohen Y. Transfer of Downy Mildew Resistance from Wild Basil (Ocimum americanum) to Sweet Basil (O. basilicum). Phytopathology 2017, 107:1-10.
Ben-Naim Y, Weitman M. Joint Action of Pb1 and Pb2 Provides Dominant Complementary Resistance Against New Races of Peronospora belbahrii (Basil Downy Mildew). Genetics and Genomics of Resistance 2022.
Itay Gonda, Adi Faigenboim, Chen Adler, Renana Milavski, Merrie-Jean Karp, Alona Shachter, Gil Ronen, Kobi Baruch, David Chaimovitsh, Nativ Dudai. The genome sequence of tetraploid sweet basil, Ocimum basilicum L., provides tools for advanced genome editing and molecular breeding (2020). DNA Research, Volume 27, Issue 5.

1. A method for producing a disease resistant cultivated basil plant, the method comprising:
a. selecting a first Ocimum americanum basil plant for having a first allele conferring basil downy mildew (BDM) race-0 resistance, wherein the first allele comprises a nucleotide sequence of SEQ ID NO:1 with a Thymine (T) at position 7, a nucleotide sequence of SEQ ID NO:2 with an Adenine (A) at position 5, a nucleotide sequence of SEQ ID NO:3 with a Cytosine (C) at position 48, a nucleotide sequence of SEQ ID NO: 4 with a Guanine (G) at position 4, a nucleotide sequence of SEQ ID NO:5 with a T at position 19, a nucleotide sequence of SEQ ID NO:6 with an A at position 8, and a nucleotide sequence of SEQ ID NO:7 with a T at position 29 resistance; and
selecting a second Ocimum americanum basil plant for having a second allele conferring basil downy mildew (BDM) race-1 resistance, wherein the second allele comprises a nucleotide sequence of SEQ ID NO:8 with a T at position 55, a nucleotide sequence of SEQ ID NO:9 with a T at position 41, a nucleotide sequence of SEQ ID NO: 10 with a C at position 11, and a nucleotide sequence of SNP of SEQ ID NO:11 with a G at position 6;
b. crossing the first BDM race-O resistant Ocimum americanum basil plant and the second BDM race-1 resistant Ocimum americanum basil plant with a BDM nonresistant Ocimum basilicum basil plant to produce a first and a second F1 plant, respectively;
c. backcrossing and optionally self-pollinating the first F1 plant, and selecting for a first BDM race-0 resistant Ocimum basilicum basil plant comprising the first allele conferring BDM race-0 resistance, and
backcrossing and optionally self-pollinating the second F1 plant, and selecting for a second BDM race-1 resistant Ocimum basilicum basil plant comprising the second allele conferring BDM race-1 resistance;
d. crossing the first and second Ocimum basilicum basil plants to produce a double BDM race-0 and race-1 resistant Ocimum basilicum basil plant, optionally backcrossing the F1 plants from the cross with a BDM nonresistant basil plant; and
e. selecting for Ocimum basilicum basil plant having resistance to BDM race-0 and BDM race-1, wherein the double resistant basil plant has introgressed into its genome the first and the second alleles, comprising the nucleotide sequence of SEQ ID NO:1 with a Thymine (T) at position 7, the nucleotide sequence of SEQ ID NO:2 with an Adenine (A) at position 5, the nucleotide sequence of SEQ ID NO:3 with a Cytosine (C) at position 48, the nucleotide sequence of SEQ ID NO:4 with a Guanine (G) at position 4, the nucleotide sequence of SEQ ID NO:5 with a T at position 19, the nucleotide sequence of SEQ ID NO:6 with an A at position 8, the nucleotide sequence of SEQ ID NO: 7 with a T at position 29 resistance, the nucleotide sequence of SEQ ID NO:8 with a T at position 55, the nucleotide sequence of SEQ ID NO:9 with a T at position 41, the nucleotide sequence of SEQ ID NO: 10 with a C at position 11, and the nucleotide sequence of SNP of SEQ ID NO:11 with a G at position 6, conferring an increased resistance to BDM race-0 and to BDM race-1, as compared to that of a basil plant comprising only the first allele or comprising only the second allele.

2. The method according to claim 1, wherein said step of crossing comprises steps of:
a. crossing the first Ocimum americanum basil plant exhibiting resistance to BDM race-0 with a BDM nonresistant Ocimum basilicum basil plant to produce first F1 plants;
b. crossing the first Ocimum americanum basil plant exhibiting resistance to BDM race-1 with a BDM nonresistant Ocimum basilicum basil plant to produce second F1 plants;
c. backcrossing and self-pollinating the first F1 plants to produce a first BDM race-0 resistant Ocimum basilicum basil plant comprising the first allele conferring the BDM race-0 resistance;
d. backcrossing and self-pollinating the second F1 plants to produce a second BDM race-1 resistant Ocimum basilicum basil plant comprising the second allele conferring the BDM race-1 resistance;
e. crossing the first and second Ocimum basilicum basil plant to produce a double BDM resistance Ocimum basilicum basil plant exhibiting an increased resistance to BDM race-0 and to BDM race-1 as compared to that of a basil plant comprising only the first allele or comprising only the second allele.

3. The method according to claim 2, wherein the method further comprises a step of analyzing the genomic sequence of the double BDM resistance Ocimum basilicum basil plant for the presence of the allele conferring BDM race-0 resistance and the allele conferring the BDM race-1 resistance.

4. The method according to claim 1, wherein the double BDM resistance Ocimum basilicum basil plant exhibits synergistic effect with respect to increased resistance to BDM race-0 and BDM race-1, as compared to that of a basil plant comprising only the first allele or comprising only the second allele.

5. The method according to claim 1, wherein said cultivated basil plant is Ocimum basilicum, and the genome of said plant comprises (a) a nucleotide sequence of SEQ ID NO:12 with a Guanine (G) at position 41, (b) a nucleotide sequence of SEQ ID NO:13 with a Thymine (T) at position 7, and (c) a nucleotide sequence of SEQ ID NO:14 with a Cytosine (C) at position 7.

6. The method according to claim 1, wherein steps a-c comprises:
pollinating a first cultivated BDM nonresistant basil plant with pollen from a first wild BDM race-resistant basil plant and pollinating a second cultivated BDM nonresistant basil plant with pollen from a second wild BDM race-1 resistant basil plant;
rescuing fertilized ovules from the first and the second cultivated pollinated basil plants;
growing the rescued fertilized ovules to produce first and second F1 plants, respectively;
backcrossing the first and the second F1 plants with the nonresistant cultivated basil plant; and
selecting for a first basil plant having resistance to BDM race-0 from the first F1 plant backcross, wherein the BDM race-0 resistant basil plant has introgressed into its genome the first allele conferring resistance to BDM race-0, and
selecting for a second basil plant having resistance to BDM race-1 from the second F1 plant backcross, wherein the BDM race-1 resistant basil plant has introgressed into its genome the second allele conferring resistance to BDM race-1.

7. A basil plant produced by the method according to claim 1.

8. The basil plant according to claim 7, wherein the plant is a plant part, optionally wherein the plant part is a seed.

9. An allele being inherited to a progeny plant, wherein said allele confers resistance to basil downy mildew (BDM) race-0 or to BDM race-1, wherein said allele comprises:
a. a first allele comprising a nucleotide sequence of SEQ ID NO:1 with a Thymine (T) at position 7, a nucleotide sequence of SEQ ID NO:2 with an Adenine (A) at position 5, a nucleotide sequence of SEQ ID NO:3 with a Cytosine (C) at position 48, a nucleotide sequence of SEQ ID NO:4 with a Guanine (G) at position 4, a nucleotide sequence of SEQ ID NO:5 with a T at position 19, a nucleotide sequence of SEQ ID NO:6 with an A at position 8, and a nucleotide sequence of SEQ ID NO: 7 with a T at position 29, said first allele is conferring BDM race-0 resistance; and
b. a second allele comprising a nucleotide sequence of SEQ ID NO:8 with a T at position 55, a nucleotide sequence of SEQ ID NO:9 with a T at position 41, a nucleotide sequence of SEQ ID NO:10 with a C at position 11, and a nucleotide sequence of SNP of SEQ ID NO:11 with a G at position 6, said second allele is conferring BDM race-1 resistance;
c. a combination of the first allele of (a) and the second allele of (b), said allele combination is conferring an increased resistance to BDM race-0 and BDM race-1 as compared to that conferred by only the first allele or only the second allele.

10. Isolated nucleotide sequences annealing with the nucleotide sequence of said allele of claim 9, wherein (a) said nucleotide sequences annealing with sequences of claim 9(a) are suitable for the detection and/or production of Ocimum basilicum basil plant exhibiting resistance to BDM race-0, (b) said nucleotide sequences annealing with sequences of claim 9(b) are suitable for the detection and/or production of Ocimum basilicum basil plant exhibiting resistance to BDM race-1, and (c) said nucleotide sequences annealing with sequences of claim 9(c) are suitable for the detection and/or production of Ocimum basilicum basil plant exhibiting increased resistance to both BDM race-0 and BDM race-1, as compared to that conferred by only the first allele or only the second allele.

11. A method for the detection of a basil plant exhibiting resistance to basil downy mildew (BDM) race-0 and to BDM race-1, the method comprises steps of identifying in the basil plant genome the presence of
a. a first allele conferring BDM race-0 resistance, wherein the first allele comprises a nucleotide sequence of SEQ ID NO: 1 with a Thymine (T) at position 7, a nucleotide sequence of SEQ ID NO:2 with an Adenine (A) at position 5, a nucleotide sequence of SEQ ID NO:3 with a Cytosine (C) at position 48, a nucleotide sequence of SEQ ID NO: 4 with a Guanine (G) at position 4, a nucleotide sequence of SEQ ID NO:5 with a T at position 19, a nucleotide sequence of SEQ ID NO:6 with an A at position 8, and a nucleotide sequence of SEQ ID NO:7 with a T at position 29 resistance; and
b. a second allele conferring BDM race-1 resistance, wherein the second allele comprises a nucleotide sequence of SEQ ID NO:8 with a T at position 55, a nucleotide sequence of SEQ ID NO:9 with a T at position 41, a nucleotide sequence of SEQ ID NO:10 with a C at position 11, and a nucleotide sequence of SNP of SEQ ID NO:11 with a G at position 6.

12. The method according to claim 11, wherein the basil plant exhibits synergistic effect with respect to increased resistance to BDM race-0 and to BDM race-1, as compared to that of a basil plant comprising only the first allele or comprising only the second allele.
